SEQUENCE LISTING (31 sequences)

SEQ ID NO: 1 - protein, 256 aa, Herpesvirus saimiri
MANEGEEIELTEFPETEKERKDEEKLSSCSEETTNTSSSSGSDHVPVPIEVNVIIQNSSRTEDELQNSKEIELTGFQGKLSSCSEETTAPSSSYSSKQASVFIEENGDNETSTYRPQNVLTNLNSLYTTFEDARAQGKGMVRHKSEDLQSFLEKYPPDFRKPKRDLSATWDPGMPTPPLPPRPANLGERQASTVRLHVKESNCKQPRERKANERNIVKDLKRLENKINVIICLVVVILAVLLLVTVLSILHIGMKS

SEQ ID NO: 2 - protein, 14 aa, synthetic construct
MEQKLISEEDLSAG

SEQ ID NO: 3 - protein, 270 aa, synthetic construct
MEQKLISEEDLSAGMANEGEEIELTEFPETEKERKDEEKLSSCSEETTNTSSSSGSDHVPVPIEVNVIIQNSSRTEDELQNSKEIELTGFQGKLSSCSEETTAPSSSYSSKQASVFIEENGDNETSTYRPQNVLTNLNSLYTTFEDARAQGKGMVRHKSEDLQSFLEKYPPDFRKPKRDLSATWDPGMPTPPLPPRPANLGERQASTVRLHVKESNCKQPRERKANERNIVKDLKRLENKINVIICLVVVILAVLLLVTVLSILHIGMKS

SEQ ID NO: 4 - protein, 270 aa, synthetic construct
MEQKLISEEDLSAGMANEGEEIELTEFPETEKERKDEEKLSSCSEETTNTSSSSGSDHVPVPIEVNVIIQNSSRTEDELQNSKEIELTGFQGKLSSCSEETTAPSSSYSSKQASVFIEENGDNETSTARPQNVLTNLNSLATTFEDARAQGKGMVRHKSEDLQSFLEKYPPDFRKPKRDLSATWDPGMPTPPLPPRPANLGERQASTVRLHVKESNCKQPRERKANERNIVKDLKRLENKINVIICLVVVILAVLLLVTVLSILHIGMKS

SEQ ID NO: 5 - protein, 164 aa, synthetic construct
MEQKLISEEDLSAGGDNETSTARPQNVLTNLNSLATTFEDARAQGKGMVRHKSEDLQSFLEKYPPDFRKPKRDLSATWDPGMPTPPLPPRPANLGERQASTVRLHVKESNCKQPRERKANERNIVKDLKRLENKINVIICLVVVILAVLLLVTVLSILHIGMKS

SEQ ID NO: 6 - protein, 257 aa, synthetic construct
MEQKLISEEDLSAGMANEGEEIELTEFPETEKERKDEEKLSSCSEETTNTSSSSGSDHVPVPIEVNVIIQNSSRTEDELQNSKEIELTGFQGKLSSCSEETTAPSSSYSSKQASVFIEENGDNETSTARPQNVLTNLNSLATTFEDARAQGKGMVRHKSEDLQSFLEKYPPDFRKPKRDLSATWDPGMPTPPLPPRPANLGERQASTVRLHVKESNCKQPRERKANERNVIICLVVVILAVLLLVTVLSILHIGMKS

SEQ ID NO: 7 - protein, 151 aa, synthetic construct
MEQKLISEEDLSAGGDNETSTARPQNVLTNLNSLATTFEDARAQGKGMVRHKSEDLQSFLEKYPPDFRKPKRDLSATWDPGMPTPPLPPRPANLGERQASTVRLHVKESNCKQPRERKANERNVIICLVVVILAVLLLVTVLSILHIGMKS

SEQ ID NO: 8 - protein, 123 aa, synthetic construct
MEQKLISEEDLSAGGDNETSTARPQNVLTNLNSLATTFEDARAQGKGMVRHKSEDLQSFLEKYPPDFRKPKRDLSATWDPGMPTPPLPPRPANLGERQASTVRLHVKESNCKQPRERKANERN

SEQ ID NO: 9 - protein, 5 aa, synthetic construct
YDKPH

SEQ ID NO: 10 - protein, 5 aa, synthetic construct
YLSLQ

SEQ ID NO: 11 - protein, 17 aa, synthetic construct
GYDKPHVLVDLLVDDSG

SEQ ID NO: 12 - protein, 17 aa, synthetic construct
DAYLSLQELQGQDPTHL

SEQ ID NO: 13 - protein, 123 aa, synthetic construct
MEQKLISEEDLSAGGDNETSTYRPQNVLTNLNSLATTFEDARAQGKGMVRHKSEDLQSFLEKYPPDFRKPKRDLSATWDPGMPTPPLPPRPANLGERQASTVRLHVKESNCKQPRERKANERN

SEQ ID NO: 14 - protein, 123 aa, synthetic construct
MEQKLISEEDLSAGGDNETSTARPQNVLTNLNSLYTTFEDARAQGKGMVRHKSEDLQSFLEKYPPDFRKPKRDLSATWDPGMPTPPLPPRPANLGERQASTVRLHVKESNCKQPRERKANERN

SEQ ID NO: 15 - protein, 123 aa, synthetic construct
MEQKLISEEDLSAGGDNETSTYRPQNVLTNLNSLYTTFEDARAQGKGMVRHKSEDLQSFLEKYPPDFRKPKRDLSATWDPGMPTPPLPPRPANLGERQASTVRLHVKESNCKQPRERKANERN

SEQ ID NO: 16 - protein, 123 aa, synthetic construct
MEQKLISEEDLSAGGDNETSTYDKPHVLTNLNSLATTFEDARAQGKGMVRHKSEDLQSFLEKYPPDFRKPKRDLSATWDPGMPTPPLPPRPANLGERQASTVRLHVKESNCKQPRERKANERN

SEQ ID NO: 17 - protein, 123 aa, synthetic construct
MEQKLISEEDLSAGGDNETSTARPQNVLTNLNSLYLSLQDARAQGKGMVRHKSEDLQSFLEKYPPDFRKPKRDLSATWDPGMPTPPLPPRPANLGERQASTVRLHVKESNCKQPRERKANERN

SEQ ID NO: 18 - protein, 123 aa, synthetic construct
MEQKLISEEDLSAGGDNETSTYDKPHVLTNLNSLYLSLQDARAQGKGMVRHKSEDLQSFLEKYPPDFRKPKRDLSATWDPGMPTPPLPPRPANLGERQASTVRLHVKESNCKQPRERKANERN

SEQ ID NO: 19 - protein, 123 aa, synthetic construct
MEQKLISEEDLSAGGDNETSTGYDKPHVLVDLLVDDSGEDARAQGKGMVRHKSEDLQSFLEKYPPDFRKPKRDLSATWDPGMPTPPLPPRPANLGERQASTVRLHVKESNCKQPRERKANERN

SEQ ID NO: 20 - protein, 123 aa, synthetic construct
MEQKLISEEDLSAGGDNETSTDAYLSLQELQGQDPTHLEDARAQGKGMVRHKSEDLQSFLEKYPPDFRKPKRDLSATWDPGMPTPPLPPRPANLGERQASTVRLHVKESNCKQPRERKANERN

SEQ ID NO: 21 - protein, 123 aa, synthetic construct
MEQKLISEEDLSAGGDNETSTYRHQNVLTNLNSLATTFEDARAQGKGMVRHKSEDLQSFLEKYPPDFRKPKRDLSATWDPGMPTPPLPPRPANLGERQASTVRLHVKESNCKQPRERKANERN

SEQ ID NO: 22 - protein, 123 aa, synthetic construct
MEQKLISEEDLSAGGDNETSTARPQNVLTNLNSLYKAFEDARAQGKGMVRHKSEDLQSFLEKYPPDFRKPKRDLSATWDPGMPTPPLPPRPANLGERQASTVRLHVKESNCKQPRERKANERN

SEQ ID NO: 23 - protein, 123 aa, synthetic construct
MEQKLISEEDLSAGGDNETSTARPQNVLTNLNSLYKPFEDARAQGKGMVRHKSEDLQSFLEKYPPDFRKPKRDLSATWDPGMPTPPLPPRPANLGERQASTVRLHVKESNCKQPRERKANERN

SEQ ID NO: 24 - protein, 123 aa, synthetic construct
MEQKLISEEDLSAGGDNTHDGYLPSNILTNLNSLYLSLQDARAQGKGMVRHKSEDLQSFLEKYPPDFRKPKRDLSATWDPGMPTPPLPPRPANLGERQASTVRLHVKESNCKQPRERKANERN

SEQ ID NO: 25 - protein, 123 aa, synthetic construct
MEQKLISEEDLSAGGDNTHDGYLPSNILTNLNSLATTFEDARAQGKGMVRHKSEDLQSFLEKYPPDFRKPKRDLSATWDPGMPTPPLPPRPANLGERQASTVRLHVKESNCKQPRERKANERN

SEQ ID NO: 26 - protein, 4 aa, synthetic construct
YRHQ

SEQ ID NO: 27 - protein, 4 aa, synthetic construct
YKAF

SEQ ID NO: 28 - protein, 4 aa, synthetic construct
YKPF

SEQ ID NO: 29 - protein, 7 aa, synthetic construct
YLPSNIL

SEQ ID NO: 30 - protein, 9 aa, synthetic construct
PTPPLPPRP

SEQ ID NO: 31 - protein, 16 aa, synthetic construct
VRHKSEDLQSFLEKYP

(19) United States
(12) Patent Application Publication
(10) Pub. No.: US 2026/0364110 A1
(43) Pub. Date: Nov. 19, 2026

(54) STAT ACTIVATORS AND METHODS OF USE FOR T-CELL THERAPY

(71) Applicant: The Regents of the University of Michigan, Ann Arbor, MI (US)

(72) Inventor: Adam Courtney, Ann Arbor, MI (US)

(21) Appl. No.: 19/489,110

(22) PCT Filed: May 31, 2024
(86) PCT No.: PCT/US2024/031878

§ 371 Date: Dec. 1, 2025

Related U.S. Application Data
(60) Provisional application No. 63/470,646, filed on Jun. 2, 2023.

(51) Int. Cl. A61K 35/17; A61K 38/00; A61K 40/11; A61K 40/32; A61K 40/42; A61P 35/00; C07K 14/47; C07K 14/715

(52) U.S. Cl. CPC A61K 35/17; A61K 40/11; A61K 40/32; A61K 40/4202; A61P 35/00; C07K 14/4705; C07K 14/7155; A61K 38/00; C07K 2319/70

(57) ABSTRACT

Provided herein activators of STAT proteins and methods of use in treating cancer. In some aspects, provided herein are bispecific peptides that bind to STAT and a kinase, and methods of use in adoptive T-cell therapy methods.

### PRIORITY STATEMENT

This application claims priority to U.S. Provisional Application No. 63/470,646, filed Jun. 2, 2023, the entire contents of which are incorporated herein by reference for all purposes.

### TECHNICAL FIELD

Provided herein are STAT activators and methods of use thereof. In particular, provided herein are bispecific peptides that bind to one or more STAT proteins and a kinase, and methods of use thereof for T-cell based cancer therapies.

### BACKGROUND

Cancer can evade T-cell mediated destruction by establishing a suppressive tumor microenvironment (TME) that deprives T-cells of pro-survival cytokines, such as IL-2. These cytokines bind to their cognate receptor to cause the JAK-STAT signaling required for T-cell survival and effector functionality. As such, the cytokine IL-2 has been directly administered to cancer patients as an immunotherapy, but poor pharmacological properties and serious adverse events have limited its therapeutic potential. Accordingly, what are needed are methods for promoting T-cell efficacy without direct administration of pro-survival cytokines such as IL-2.

### SUMMARY

In some aspects, provided herein are bispecific peptides comprising at least one signal transducer and activator of transcription (STAT) binding region and at least one kinase binding region. In some embodiments, the at least one kinase binding region binds to a tyrosine kinase. In some embodiments, the at least one kinase binding region binds to an SRC family kinase. In some aspects, provided herein are bispecific peptides comprising at least one signal transducer and activator of transcription (STAT) binding region and at least one SRC family kinase binding region. In some embodiments, the at least one kinase binding region binds lymphocyte-specific protein tyrosine kinase (LCK). In some embodiments, the bispecific peptide comprises at least one STAT binding region and at least one LCK binding region. In some embodiments, the at least one STAT binding region comprises an amino acid sequence derived from one or more cytokine receptors. For example, in some embodiments the at least one STAT binding region comprises an amino acid sequence derived from an interleukin-2 receptor (IL-2R) and/or derived from an interferon gamma receptor (IFNgR).

In some embodiments, the at least one STAT binding region comprises one or more of YDKPH (SEQ ID NO: 9), YLSLQ (SEQ ID NO: 10), YRHQ (SEQ ID NO: 26), YKAE (SEQ ID NO: 27), YKPF (SEQ ID NO: 28), and YLPSNIL (SEQ ID NO: 29). In some embodiments the at least one STAT binding region comprises two of YDKPH (SEQ ID NO: 9), YLSLQ (SEQ ID NO: 10), YRHQ (SEQ ID NO: 26), YKAF (SEQ ID NO: 27), YKPF (SEQ ID NO: 28), and YLPSNIL (SEQ ID NO: 29). For example, in some embodiments the at least one STAT binding region comprises SEQ ID NO: 29 and SEQ ID NO: 10.

In some embodiments, the at least one STAT binding region comprises a sequence having at least 80% identity (e.g. at least 80%, at least 85%, at least 90%, at least 95% identity) to GYDKPHVLVDLLVDDSG (SEQ ID NO: 11). In some embodiments, the at least one STAT binding region comprises the sequence of SEQ ID NO: 11.

In some embodiments, the at least one STAT binding region comprises a sequence having at least 80% identity (e.g. at least 80%, at least 85%, at least 90%, at least 95% identity) to DAYLSLQELQGQDPTHL (SEQ ID NO: 12). In some embodiments, the at least one STAT binding region comprises the sequence of SEQ ID NO: 12.

In some embodiments, the at least one LCK binding region comprises an SH3 binding domain having at least 55% sequence identity to PTPPLPPRP (SEQ ID NO: 30) and/or a CSK homology domain having at least 50% sequence identity to VRHKSEDLQSFLEKYP (SEQ ID NO: 31). In some embodiments, the at least one LCK binding region comprises an SH3 binding domain having at least 65% sequence identity to SEQ ID NO: 30 and/or a CSK homology domain having at least 75% sequence identity to SEQ ID NO: 31. In some embodiments, the at least one LCK binding region comprises an SH3 binding domain having at least 75% sequence identity to SEQ ID NO: 30 and/or a CSK homology domain having at least 87.5% sequence identity to SEQ ID NO: 31. In some embodiments, the at least one LCK binding region comprises an SH3 binding domain having at least 85% sequence identity to SEQ ID NO: 30 and/or a CSK homology domain having at least 93.5% sequence identity to SEQ ID NO: 31. In some embodiments, the at least one LCK binding region comprises an SH3 binding domain comprising the sequence of SEQ ID NO: 30 and/or a CSK homology domain comprising the sequence of SEQ ID NO: 31.

In some aspects, provided herein are vectors encoding a bispecific peptide described herein. In some aspects, provided herein are cells expressing bispecific peptide or a vector encoding the same, as described herein. In some embodiments, the cell is an immune cell. For example, in some embodiments the cell is an immune cell such as a natural killer (NK) cell, a T-cell, a B-cell, a macrophage, or a dendritic cell. In some embodiments, the cell is a T-cell.

The bispecific peptides, vectors, and cells provided herein find use in treatment methods involving T-cell therapy. For example, in some embodiments the bispecific peptides, vectors, and cells provided herein find use in T-cell therapies for treatment of diseases including cancer, autoimmune disease, and inflammatory disease. In some aspects, provided herein is a method of treating cancer in a subject, the method comprising providing to the subject a T-cell expressing a bispecific peptide comprising at least one signal transducer and activator of transcription (STAT) binding region and at least one lymphocyte-specific protein tyrosine kinase (LCK) binding region, as described herein. In some embodiments, the T-cell expressing the bispecific peptide is co-administered to the subject in combination with exogenous IL-2. In some embodiments, the T-cell expressing the bispecific peptide is administered to the subject without co-administration of exogenous IL-2.

### BRIEF DESCRIPTION OF THE DRAWINGS

FIG. 1 shows identification of a minimal Tyrosine kinase interacting protein (TIP) that binds to LCK kinase, which is derived from the Herpesvirus saimiri. FIG. 1A is a schematic showing exemplary cell signaling pathways that may contribute to efficacy of a bispecific peptide that binds to STAT and LCK kinase, as described herein. FIG. 1B is a schematic showing different peptides generated for evaluation. Each peptide comprises the minimal TIP viral protein. Each peptide further comprises a Myc purification tag. Additional components of the various peptides are identified in FIG. 1B. FIG. 1C shows expression of the various components of each peptide as confirmed by western blot using Jurkat T-cell lysates. FIG. 1D shows LCK kinase activation by the TIP minimal viral protein. Cells were transfected with an empty vector (control) or the TIP minimal viral protein (protein 6, also referred to as “LCK activator”). LCK kinase activation was assessed by autophosphorylation (pY394). As shown in FIG. 1D, the amount of pY394 was enhanced in cells transfected with the TIP minimal viral protein compared to cells transfected with the empty vector. Expression was normalized against GAPDH. Taken together, these results demonstrate that the TIP minimal viral protein increases LCK kinase activity in CD8+ T-cells.

FIG. 2A shows a comparison of STAT binding sequences derived from cytokine receptors inserted into the minimal viral peptide. The viral STAT binding sites broadly activate many STAT proteins. The STAT binding sites from endogenous cytokine receptors can be used to target specific STATs for activation. Note the IL-2 receptor STAT binding site activates STAT5 and the IFNgR STAT binding sites activates STAT1. The size of the STAT binding region was compared (4 amino acids versus 10-12 amino acids). FIG. 2B shows STAT activation assessed by assessing phosphorylation using western blot. Jurkat T-cell lysates were used. Jurkat T-cells are a human T-cell line.

FIG. 3A shows a schematic of an exemplary cytokine-dependent mechanism of action for the JAK/STAT signaling pathway and an exemplary cytokine-independent mechanism for the bispecific peptides provided herein. FIG. 3B shows activation of various STAT proteins for the different peptides generated herein, identifying novel STAT activation profiles for the different peptides. STAT activation as tested for the WT minimal TIP viral protein, LCK kinase only, viral stat (A), viral STAT (B), viral stat (AB), an activator of STAT 1 (aSTAT1), an activator of STAT 5 (aSTAT5), an activator of STAT 1 and STAT 5 (aSTAT1/5), and an empty vector as a control, aSTAT1 is peptide 1 from FIG. 2, aSTAT5 is peptide 2 from FIG. 2, and a STAT1/5 is peptide 3 from FIG. 2. FIG. 3C summarizes the STAT activation profile for each tested peptide in a color-coded graph.

FIG. 4A shows exemplary mutations to CSKH and SH3B regions made to assess the effect on LCK activity and binding. LCK autophosphorylation (a readout of kinase activity) in mutant peptides is shown in FIG. 4B. Immunoprecipitation/pulldown was performed to detect association (binding) of minimal TIP peptide to LCK kinase. Results are shown in FIG. 4C.

An schematic of an exemplary experiment conducted herein to evaluate efficacy of aSTAT5 ex vivo using primary T-cells is shown in FIG. 5A. CD8+ T-cells were transduced with aSTAT5 or with empty constructs as a control, and cell survival and expansion was assessed in the presence and absence of exogenous IL-2 added to cell culture media. Results are shown in FIG. 5B. A schematic of an exemplary experiment conducted herein to investigate whether aSTAT5 expressing T-cells retain cell function is shown in FIG. 5C. T-cell function was assessed by the activation reporter Nur77-GFP and the intracellular cytokine IFNg. CD8+ T-cells were transduced with the aSTAT5 construct and the activation reporter Nur77-GFP. Antigen presenting cells (splenocytes) and an antigenic peptide (OVA peptide) or a control peptide (VSV peptide) were added to the culture. T-cell function was investigated in the presence and absence of exogenous IL-2. Results are shown in FIG. 5D.

A schematic of an exemplary experiment to further evaluate T-cell function is shown in FIG. 6A. T-cells transduced with the aSTAT5 construct and the activation reporter Nur77-GFP (OT-1 Nur77-GFP cells, or “OT-1” cells), were co-cultured with MC38-OVA cancer cells. T-cell-medicated killing of cancer cells was quantified by imaging cytometry (quantification of propidium iodide positive (PI+) cells) and T-cell activation was evaluated by quantifying GFP positive cells. Results are shown in FIG. 6B and FIG. 6C.

FIG. 7 shows assessment of CD8+ T-cell differentiation. CD8+ T-cells were transduced with aSTAT5, as described above, or treated with exogenous IL-2, and gene expression profiles were investigated.

A schematic showing an exemplary experiment conducted in-vivo to evaluate aSTAT5 activator efficacy in a tumorigenic mouse model is shown in FIG. 8A. Tumor antigen-specific CD8+ T-cells were transduced with aSTAT5, LCK activator (TIP minimal protein, without the STAT binding sequence), PBS, or an empty vector and treated with exogenous IL-2. C57BL/6J immunocompetent mice were treated subcutaneously with B16-OVA cells at day −10. On day 0, adoptive cell transfer was conducted using the generated aSTAT5 expressing or control tumor antigen-specific CD8+ T-cells. Tumor volume was evaluated from days 1 to 14 after adoptive cell transfer. Results are shown in FIG. 8B. At day 16, tumors were harvested and weighed. Images of harvested tumors are shown in FIG. 8D, and weights are shown in FIG. 8C.

FIG. 9A shows a schematic of an exemplary experiment conducted using a different mouse model from that of FIG. 8. Tumor antigen-specific CD8+ T-cells were transduced with aSTAT5, LCK activator (TIP minimal protein, without the STAT binding sequence), PBS, or an empty vector and treated with exogenous IL-2. C57BL/6J immunocompetent mice were treated subcutaneously with EL4-OVA cells at day −5. At day 0, ACT was conducted. Tumor size was evaluated from days 0 to 22 post ACT. Results are shown in FIG. 9B. Plots showing individual mouse responses to each treatment are shown in FIG. 9C. Overall survival plots are shown in FIG. 9D.

FIG. 10A-10H further evaluate T-cell functionality in tumors due to STAT5 activation, related to FIG. 9. FIG. 10A shows that levels of Granzyme B, a marker of T-cell functionality, was increased in tumors after STAT5 activation. FIG. 10B shows that levels of PD-1, associate with loss of T-cell function, were reduced in tumors after STAT5 activation. Results are quantified in FIG. 10B. FIGS. 10C, 10D, 10E, and 10F show results of similar experiments 5 days after ACT, demonstrating that increased granzyme B levels and decreased PD-1 levels are attributable to the STAT5 activator. FIG. 10G shows that STAT5 activation enhances CD8+ T-cell persistence in tumors, as demonstrated by increased percentage of CD45+ white blood cells following ACT with aSTAT5 vs. ACT with empty vector control. Results are quantified in FIG. 10H.

FIG. 11A shows constructs targeting STAT3, STAT 4/5, and STAT 6. Activation of STAT1, 3, 4, 5, and 6 using the constructs was assessed by western blot, as shown in FIG. 11B. FIG. 11C summarizes the STAT activation profile for each tested peptide in a color-coded graph.

### DETAILED DESCRIPTION

Section headings as used in this section and the entire disclosure herein are merely for organizational purposes and are not intended to be limiting.

All publications, patent applications, patents and other references mentioned herein are incorporated by reference in their entirety.

### 1. DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies, or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, the terms “co-administration” and variations thereof refer to the administration of at least two agent(s) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. In some embodiments, when agents or therapies are co-administered, the respective agents or therapies are administered at lower dosages than appropriate for their administration alone. Accordingly, co-administration may be especially desirable in embodiments where the co-administration of two or more agents results in sensitization of a subject to beneficial effects of one of the agents via co-administration of the other agent.

The term “carrier” as used herein refers to any pharmaceutically acceptable solvent of agents that will allow a therapeutic composition to be administered to the subject. A “carrier” as used herein, therefore, refers to such solvent as, but not limited to, water, saline, physiological saline, oil-water emulsions, gels, or any other solvent or combination of solvents and compounds known to one of skill in the art that is pharmaceutically and physiologically acceptable to the recipient human or animal. The term “pharmaceutically acceptable” as used herein refers to a compound or composition that will not impair the physiology of the recipient human or animal to the extent that the viability of the recipient is compromised. For example, “pharmaceutically acceptable” may refer to a compound or composition that does not substantially produce adverse reactions, e.g., toxic, allergic, or immunological reactions, when administered to a subject.

As used herein, the terms “subject” and “patient” are used interchangeably herein and refer to both human and nonhuman animals. The term “nonhuman animals” includes all vertebrates, e.g., mammals and non-mammals, such as nonhuman primates, sheep, dogs, cats, horses, cows, chickens, amphibians, reptiles, and the like. In some embodiments, the subject is a human. In some embodiments, the subject is a male. In some embodiments, the subject is a female. In some embodiments, the subject is suffering from cancer.

As used herein, “treat”, “treating”, “treatment”, and variations thereof refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder, or condition. In some embodiments, treating a cancer refers to the management and care of the subject for combating and reducing one or more symptoms of the cancer. For example, treating cancer may reduce tumor burden (e.g. reduce the size of one or more tumors in the subject afflicted with cancer and/or reduce the overall number of tumors in the subject afflicted with cancer). Treating a cancer may reduce or completely eliminate the cancer (e.g. completely eliminate the tumor) in the subject.

### 2. STAT ACTIVATORS

CD8+ T-cells can recognize and kill cancer cells, but can become dysfunctional within the tumor. One reason for this dysfunction is a lack of cytokines needed by CD8+ T-cells. These cytokines (such as IL-2) activate STAT proteins, such as STAT5, to keep CD8+ T-cells alive and maintain their anti-tumor function, but in the absence of IL-2, CD8+ T-cells lose their anti-tumor capabilities. Members of the signal transducer and activator of transcription (STAT) protein family are intracellular transcription factors that mediate may aspects of cellular immunity, proliferation, apoptosis, and differentiation. STAT proteins are primarily activated by membrane receptor-associated Janus kinases (JAK). Dysregulation of this pathway is frequently observed in primary tumors and leads to increased angiogenesis and immunosuppression, which enhances the survival of tumors. Currently identified STAT family proteins include STAT1, STAT2, STAT3, STAT4, STAT5 (STAT5A and STAT5B) and STAT6.

Provided herein are STAT activators that activate one or more STAT proteins and sustain the survival and function of T-cells. In some embodiments, the STAT activators sustain the survival and function of T-cells including in the absence of cytokines, such as IL-2. In some embodiments, STAT activators are peptides. In some embodiments, STAT activators are bispecific peptides that bind to a STAT protein and a kinase. In some embodiments, STAT activators are bispecific peptides that bind to a STAT protein and an SRC family kinase. In some embodiments, the STAT activators are bispecific peptides that bind to a STAT protein and a lymphocyte-specific protein tyrosine kinase (LCK). In some embodiments, provided herein is a STAT activator that sustains the survival and function of CD8+ T-cells, including the absence of cytokines such as IL-2. A viral protein was engineered to generate a minimal peptide that binds to a protein tyrosine kinase and a STAT protein. Without wishing to be bound by theory, it is hypothesized that by bringing the kinase into proximity with the STAT, the kinase can phosphorylate and activate the STAT. The STAT activators provided herein can be incorporated into primary CD8+ T-cells by viral transduction or any other desirable method. T-cells modified in this way can survive in culture without IL-2. These modified cells can recognize and kill cancer cells ex vivo under conditions where unmodified T-cells die off. Accordingly, the STAT activators can be combined with T-cell therapies where T-cells are modified ex vivo and transferred back into a patient for use in methods of treating cancer.

In some aspects, provided herein is a bispecific peptide comprising at least one signal transducer and activator of transcription (STAT) binding region and at least one kinase binding region. In some embodiments, the at least one kinase binding region binds to one or more SRC family kinases. The SRC family kinases refer to a family of non-receptor tyrosine kinases that interact with many cellular cytosolic, nuclear, and membrane proteins. The SRC family kinases include the SrcA subfamily (Src, Yes, Fyn, and Fgr) and the SrcB subfamily (Lck, Hek, Bik, and Lyn), and Frk in its own subfamily. In some embodiments, the kinase binding region binds to an SrcB subfamily kinase. In some embodiments, the at least one kinase binding region binds to lymphocyte-specific tyrosine kinase (LCK). In some embodiments, the at least one kinase binding region binds to tyrosine-protein kinase HCK (HCK). In some embodiments, the at least one kinase binding region binds to tyrosine-protein kinase BLK, also known as B lymphocyte kinase (BCK). In some embodiments, the at least one kinase binding region binds to tyrosine-protein kinase Lyn (Lyn). Binding to a given kinase does not necessarily mean that the binding is exclusive to that kinase. In some embodiments, a bispecific peptide provided herein binds to multiple kinases. For example, in some embodiments a bispecific peptide provided herein binds to multiple SRC family kinases. In some embodiments, provided herein is a bispecific peptide comprising at least one SRC family kinase binding region. In some embodiments, the at least one SRC family kinase binding region comprises at least one LCK kinase binding region (e.g. the bispecific peptide binds to an LCK kinase). Such a bispecific peptide comprising an LCK binding region indicates that the bispecific peptide binds to an LCK kinase but does not necessarily indicate that the peptide does not bind to other SRC family kinases. For example, a bispecific peptide wherein the at least one SRC family kinase binding region comprises an LCK kinase binding region may bind to the LCK kinase and one or more other SRC family kinases.

In some embodiments, the bispecific peptide comprises at least one STAT binding region and at least one lymphocyte-specific protein tyrosine kinase (LCK) binding region. The at least one STAT binding region may bind to any STAT protein or any combination of STAT proteins, including STAT1, STAT2, STAT3, STAT4, STAT5, and/or STAT6. In some embodiments, the at least one STAT binding region comprises an amino acid sequence derived from one or more cytokine receptors. For example, in some embodiments the at least one STAT binding region comprises an amino acid sequence derived from an interleukin-2 receptor (IL-2R), an interleukin-4 receptor (IL-4R), an interleukin-6 receptor (IL-6R), an interleukin-12 receptor (IL-12R), and/or derived from an interferon gamma receptor (IFNgR).

In some embodiments, the at least one STAT binding region comprises 2-30 amino acids. In some embodiments, the at least one STAT binding region comprises 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids derived from the amino acid sequence of one or more cytokine receptors. In some embodiments, the at least one STAT binding region comprises a minimal peptide sequence, such as peptide sequence of less than 10 amino acids, derived from one or more cytokine receptors.

In some embodiments, the at least one STAT binding region comprises one or more of YDKPH (SEQ ID NO: 9), YLSLQ (SEQ ID NO: 10), YRIQ (SEQ ID NO: 26), YKAF (SEQ ID NO: 27), YKPF (SEQ ID NO: 28), and YLPSNIL (SEQ ID NO: 29). In some embodiments, the at least one STAT binding region comprises SEQ ID NO: 9. In some embodiments, the at least one STAT binding region comprises SEQ ID NO: 10. In some embodiments, the at least one STAT binding region comprises SEQ ID NO: 26. In some embodiments, the at least one STAT binding region comprises SEQ ID NO: 27. In some embodiments, the at least one STAT binding region comprises SEQ ID NO: 28. In some embodiments, the at least one STAT binding region comprises SEQ ID NO: 29.

In some embodiments, the at least one STAT binding region is selected to achieve the desired activation of a specific STAT protein (e.g. STAT1, STAT2, STAT3. STAT4. STAT5, or STAT6). In some embodiments, a bispecific peptide comprising the STAT binding region of SEQ ID NO: 9 preferentially binds to and activates STAT1. In some embodiments, a bispecific peptide comprising the STAT binding region of SEQ ID NO: 10 preferentially binds to and activates STAT5. In some embodiments, a bispecific peptide comprising the STAT binding region of SEQ ID NO: 26 preferentially binds to and activates STAT3. In some embodiments, a bispecific peptide comprising the STAT binding region of SEQ ID NO: 27 preferentially binds to and activates STAT6. In some embodiments, a bispecific peptide comprising the STAT binding region of SEQ ID NO: 28 preferentially binds to and activates STAT6. In some embodiments, a bispecific peptide comprising the STAT binding region of SEQ ID NO: 29 preferentially binds to and activates STAT4.

In some embodiments the at least one STAT binding region comprises two or more of YDKPH (SEQ ID NO: 9), YLSLQ (SEQ ID NO: 10), YRHQ (SEQ ID NO: 26), YKAF (SEQ ID NO: 27), YKPF (SEQ ID NO: 28), and YLPSNIL (SEQ ID NO: 29). Any combination of two or more of SEQ ID NO: 9. SEQ ID NO: 10, SEQ ID NO: 26, SEQ ID NO: 27, SEQ ID NO: 28, and SEQ ID NO: 29 may be present in the bispecific peptide to achieve the desired STAT activation profile. In some embodiments, the bispecific peptide comprises two STAT binding regions, three STAT binding regions, four STAT binding regions, or five or more STAT binding regions. In some embodiments, two or more STAT binding regions are included in the bispecific peptide to preferentially bind to an activate multiple STAT proteins. For example, in some embodiments, the at least one STAT binding region comprises SEQ ID NO: 9 and SEQ ID NO: 10. A bispecific peptide comprising the STAT binding region of SEQ ID NO: 9 and the STAT binding region of SEQ ID NO: 10 preferentially binds to STAT1 and STAT5. As another example, in some embodiments the at least one STAT binding region comprises SEQ ID NO: 10 and SEQ ID NO: 29. A bispecific peptide comprising the STAT binding region of SEQ ID NO: 10 and the STAT binding region of SEQ ID NO: 29 preferentially binds to STAT4 and STAT5.

In some embodiments, when a bispecific peptide has multiple STAT binding regions each STAT binding region is separated by a suitable number of amino acids. In some embodiments, each STAT binding region is separated by a sequence of 5-15 amino acids. For example, in some embodiments each STAT binding region is separated by a sequence of 5, 6, 7, 8, 9, 10, 11, 12, 13, 14, or 15 amino acids. In some embodiments, each STAT binding region is separated by a sequence of 8 amino acids.

In some embodiments, the at least one STAT binding region comprises a sequence having at least 80% identity (e.g. at least 80%, at least 85%, at least 90%, at least 95% identity) to GYDKPHVLVDLLVDDSG (SEQ ID NO: 11). In some embodiments, the at least one STAT binding region comprises the sequence of SEQ ID NO: 11. Such a bispecific peptide may preferentially bind to an activate STAT1.

In some embodiments, the at least one STAT binding region comprises a sequence having at least 80% identity (e.g. at least 80%, at least 85%, at least 90%, at least 95% identity) to DAYLSLQELQGQDPTHL (SEQ ID NO: 12). In some embodiments, the at least one STAT binding region comprises the sequence of SEQ ID NO: 12. Such a bispecific peptide may preferentially bind to an activate STAT5.

In some embodiments, the bispecific peptide comprises at least one LCK binding region. In some embodiments, the at least one LCK binding region comprises an SH3 binding domain having at least 55% sequence identity to PTPPLPPRP (SEQ ID NO: 30) and/or a CSK homology domain having at least 50% sequence identity to VRHKSEDLQSFLEKYP (SEQ ID NO: 31). In some embodiments, the at least one LCK binding region comprises an SH3 binding domain having at least 65% sequence identity to SEQ ID NO: 30 and/or a CSK homology domain having at least 75% sequence identity to SEQ ID NO: 31. In some embodiments, the at least one LCK binding region comprises an SH3 binding domain having at least 75% sequence identity to SEQ ID NO: 30 and/or a CSK homology domain having at least 87.5% sequence identity to SEQ ID NO: 31. In some embodiments, the at least one LCK binding region comprises an SH3 binding domain having at least 85% sequence identity to SEQ ID NO: 30 and/or a CSK homology domain having at least 93.5% sequence identity to SEQ ID NO: 31. In some embodiments, the at least one LCK binding region comprises an SH3 binding domain comprising the sequence of SEQ ID NO: 30 and/or a CSK homology domain comprising the sequence of SEQ ID NO: 31.

In some embodiments, the bispecific peptide comprises at least one LCK binding region. In some embodiments, the at least one LCK binding region comprises an SH3 binding domain having at least 55% sequence identity to PTPPLPPRP (SEQ ID NO: 30) and a CSK homology domain having at least 50% sequence identity to VRHKSEDLQSFLEKYP (SEQ ID NO: 31). In some embodiments, the at least one LCK binding region comprises an SH3 binding domain having at least 65% sequence identity to SEQ ID NO: 30 and a CSK homology domain having at least 75% sequence identity to SEQ ID NO: 31. In some embodiments, the at least one LCK binding region comprises an SH3 binding domain having at least 75% sequence identity to SEQ ID NO: 30 and a CSK homology domain having at least 87.5% sequence identity to SEQ ID NO: 31. In some embodiments, the at least one LCK binding region comprises an SH3 binding domain having at least 85% sequence identity to SEQ ID NO: 30 and a CSK homology domain having at least 93.5% sequence identity to SEQ ID NO: 31. In some embodiments, the at least one LCK binding region comprises an SH3 binding domain comprising the sequence of SEQ ID NO: 30 and a CSK homology domain comprising the sequence of SEQ ID NO: 31.

In some embodiments, the bispecific peptide comprises an amino acid sequence having at least 80% sequence identity (e.g. at least 80%, at least 85%, at least 90%, at least 91%, at least 92%, at least 93%, at least 94%, at least 95%, at least 96%, at least 97%, at least 98%, at least 99% identity) to SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, or SEQ ID NO: 25. In some embodiments, the bispecific peptide comprises an amino acid sequence having at least 80% sequence identity (e.g. at least 80%, at least 85%, at least 90%, at least 91%, at least 92%, at least 93%, at least 94%, at least 95%, at least 96%, at least 97%, at least 98%, at least 99% identity) to SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, or SEQ ID NO: 25 without the Myc tag sequence (MEQKLISEEDLSAG, SEQ ID NO: 2) (e.g. the Myc tag sequence is removed from SEQ ID NO: 16-25, and the bispecific peptide has 80% sequence identify with the resulting, untagged sequence).

In some embodiments, the bispecific peptide has a total length of less than 200 amino acids. In some embodiments, the bispecific peptide comprises 50-200 amino acids. In some embodiments, the bispecific peptide comprises 40-200 amino acids, 40-190 amino acids, 50-180 amino acids, 50-170 amino acids, 60-160 amino acids, 70-150 amino acids, 80-140 amino acids, 90-130 amino acids, or 100-125 amino acids.

In some embodiments, the bispecific peptide binds to (e.g. activates) one or more STAT proteins. The STAT family of proteins comprises STAT1, STAT2, STAT3, STAT4, STAT5, and STAT6. In some embodiments, the bispecific peptide activates STAT1. In some embodiments, the bispecific peptide activates STAT3. In some embodiments, the bispecific peptide activates STAT4. In some embodiments, the bispecific peptide activates STAT5. In some embodiments, the bispecific peptide activates STAT1 and STAT5. In some embodiments, the bispecific peptide activates STAT4 and STAT5. In some embodiments, the bispecific peptide activates STAT1 and STAT6.

In some aspects, provided herein are vectors encoding a STAT activator. In some embodiments, provided herein are vectors encoding a bispecific peptide described herein. In some embodiments, the construct is a vector. Suitable vectors include viral vectors (e.g. retroviruses, adenoviruses, adeno-associated viruses, lentiviruses, herpes simplex viruses) and non-viral vectors (e.g. plasmids, liposomes, etc.). In some embodiments, the STAT activator is encoded by DNA. In some embodiments, the STAT activator is encoded by RNA.

In some aspects, provided herein are cells expressing a STAT activator as described herein. In some embodiments, provided herein are cells expressing a bispecific peptide described herein. In some embodiments, provided herein are cells expressing a vector encoding a bispecific peptide described herein. In some embodiments, the bispecific peptides described herein are directly introduced into cells, such as via protein transduction methods using protein transduction domains, cell penetrating peptides, and the like. In some embodiments, vectors encoding a bispecific peptide are transduced into cells. In some embodiments, bispecific peptides are introduced into cells via suitable techniques such as electroporation, microinjection, or use of chemicals to selectively permeabilize the cell membrane.

In some embodiments, the cell (e.g. the cell expressing the bispecific peptide or vector encoding the bispecific peptide) is an immune cell. The term “immune cell” is inclusive of lymphocytes (e.g. T-cells, B-cells), natural killer cells, granulocytes (basophils, eosinophils, neutrophils), masT-cells, monocytes, and macrophages. In some embodiments, the cell is a T-cell. In some embodiments, the cell is a CD8+ T-cell. In some embodiments, the cell is a CD4+ T-cell. In some embodiments, the cell is an antigen-specific cell. In some embodiments, the cell is an antigen-specific T-cell.

The STAT activators (e.g. bispecific peptides), vectors, and cells described herein find use in a variety of methods, including methods of promoting survival and function of immune cells. In some embodiments, the STAT activators, vectors, and cells described herein find use in promoting survival and function of T-cells, such as those used in adoptive cell transfer therapy. Adoptive cell transfer (ACT) therapy, also referred to as T-cell transfer therapy, refers to a type of immunotherapy involving collecting immune cells from a subject, in some cases modifying the cells in a suitable manner, expanding the number of immune cells using laboratory techniques (e.g. increasing the number of immune cells), and then providing the cells back to the subject (e.g. by injection). In some aspects, the STAT activators (e.g. bispecific peptides), vectors, and cells described herein find use of promoting survival and function of T-cells used in adoptive cell therapy. In some embodiments, the STAT activators (e.g. bispecific peptides), vectors, and cells described herein find use in methods of treating a variety of diseases, including cancer or autoimmune disease in a subject, such as through adoptive cell therapy. In some embodiments, methods of promoting T-cell survival and/or methods of treating cancer or autoimmune disease in a subject comprise providing to the subject a T-cell expressing a bispecific peptide comprising a signal transducer and activator of transcription (STAT) binding region and a lymphocyte-specific protein tyrosine kinase (LCK) binding region, as described herein. In some embodiments, the T-cell is co-administered to the subject with exogenous IL-2. In some embodiments, the T-cell is co-administered with a lower dose of exogenous IL-2 than would otherwise be required for methods of promoting T-cell function and survival using exogenous IL-2 alone. Accordingly, the use of such T-cells can be used in conjunction with low doses of exogenous IL-2 to promote T-cell function and survival as an anti-cancer therapy, without toxicities otherwise observed with higher doses of exogenous IL-2. In some embodiments, the T-cell is administered to the subject without exogenous IL-2.

Exemplary adoptive cell therapy methods, such as for treating cancer, inflammatory disease, and/or autoimmune disease, involve use of tumor-infiltrating lymphocytes (TILs) and chimeric antigen receptor (CAR) T-cells, which refer to T-cells modified to express chimeric antigen receptors on their surface, improving their ability to recognize and attack cancerous cells. The STAT activators and vectors encoding the same described herein can be used in conjunction with any suitable T-cell, including TILs. CAR-T-cells. CD8+ T-cells, CD4+ T-cells, etc.

In some embodiments, the subject has or is at risk of having cancer. The term “cancer” is used in the broadest sense and refers to a disease characterized by uncontrolled division of abnormal cells in a part of the body. The cancer may be any cancer, including carcinomas, sarcomas, lymphomas, leukemias, and myelomas. In some embodiments, the subject is a vertebrate. In some embodiments, the subject is a mammal. In some embodiments, the subject is a human.

### EXAMPLES

### Example 1

Experiments were conducted herein to evaluate whether cytokine-independent activation of STAT5 enables CD8+ T-cells to persist ex vivo under suppressive culture conditions. Provided herein is a direct activator of STAT5 that allows CD8+ T-cells to remain viable in the absence of IL-2 over several days. The STAT5 activator allows CD8+ T-cells to retain their functionality, and their capacity to become activated by antigen and exert T-cell-mediated cytotoxicity. Specifically, experiments conducted herein demonstrate that under suppressive culture conditions a STAT5 activator allowed CD8+ T-cells to become activated, as assessed by upregulation the Nur77-GFP reporter, and kill MC38-OVA cancer cells. Direct activation of STAT5 was also sufficient for the production of IFNg upon restimulation by antigen presenting cells. These data demonstrate that CD8+ T-cell survival and cytotoxic capacity can be maintained in cytokine-depleted environments by direct activation of STAT5. A STAT5 activator was transduced into CD8+ T-cells and tumor-specific T-cells were adoptively transferred into tumor-bearing mice. Using the EL4-OVA model, CD8+ T-cells modified with a STAT5 activator demonstrated potent tumor regression and reduced tumor outgrowth following adoptive cell transfer (ACT). These findings highlight that T-cells can be improved to resist the miscommunication that cancer causes by targeting essential intracellular signaling pathways needed for a sustained anti-tumor response.

A minimal tyrosine-protein kinase-interacting protein (TIP) viral protein that binds to LCK kinase was identified, and various peptides were generated containing the minimal TIP viral protein. The complete sequence of the TIP viral protein used to identify the minimal TIP viral protein is:

(SEQ ID NO: 1)
MANEGEEIELTEFPETEKERKDEEKLSSCSEETTNTSSSSGSDHVPVPI
EVNVIIQNSSRTEDELQNSKEIELTGFQGKLSSCSEETTAPSSSYSSKQ
ASVFIEENGDNETSTYRPQNVLTNLNSLYTTFEDARAQGKGMVRHKSED
LQSFLEKYPPDFRKPKRDLSATWDPGMPTPPLPPRPANLGERQASTVRL
HVKESNCKQPRERKANERNIVKDLKRLENKINVIICLVVVILAVLLLVT
VSILHIGMKS.

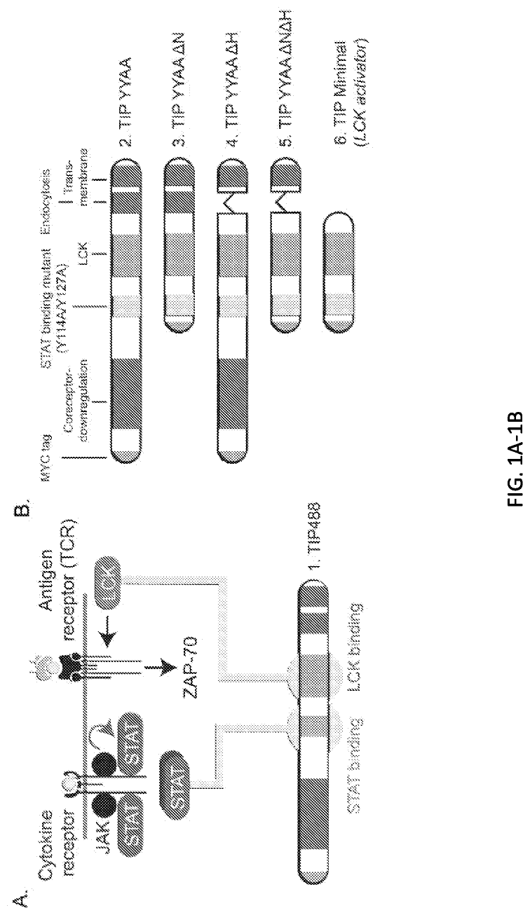

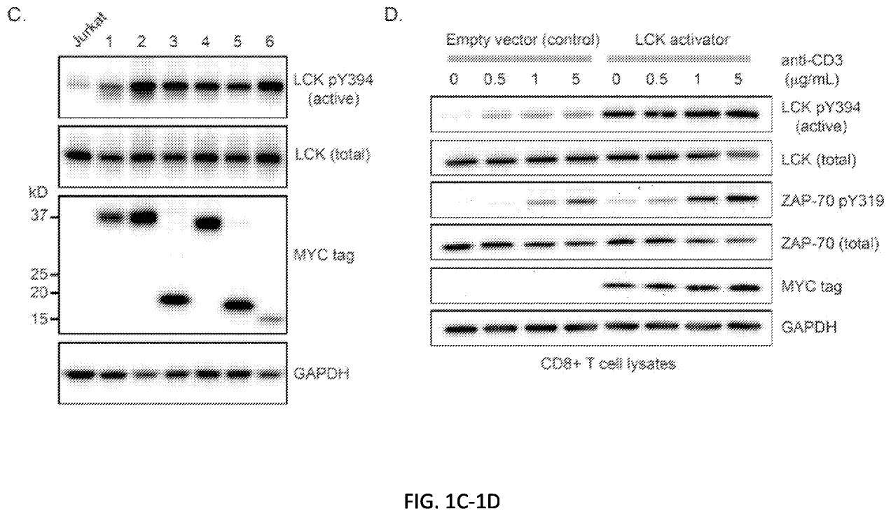

Different peptides containing the minimal TIP viral protein are shown in FIG. 1B. The sequences of the generated peptides are shown below, with numbers corresponding to the number shown in FIG. 1B. The TIP regions are annotated as follows:
MYC tag—MEQKLISEEDL SAG (SEQ ID NO: 2)
LCK interaction sites: CSKH, SH3B
STAT binding site tyrosine residues A[Y114] and B[Y127] are italicized and underlined). Y to A mutations are similarly identified to denote position of mutated residue.

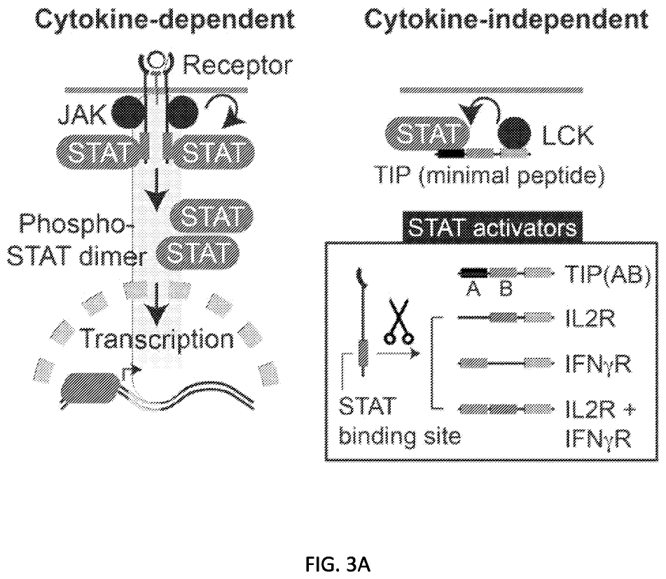

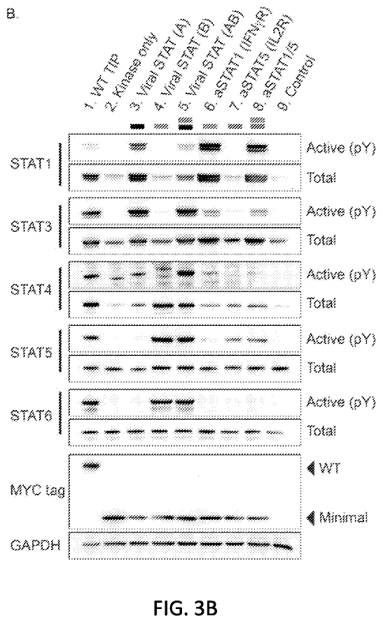

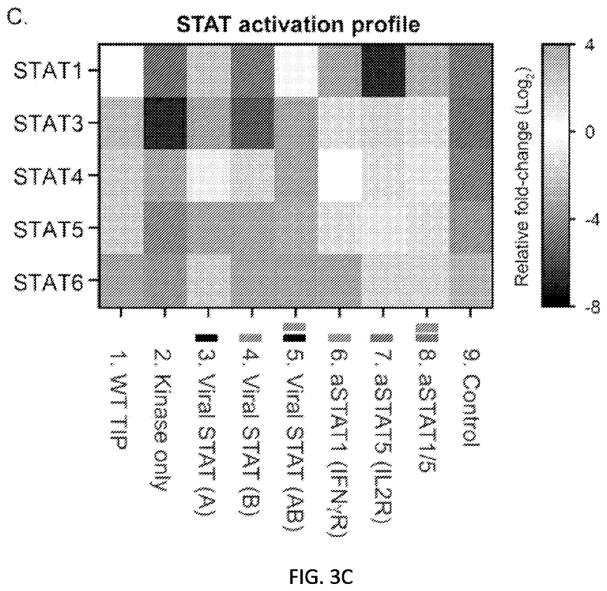

### 1. Full Length TIP488+MYC Tag [Construct 1 FIG. 3B]

(SEQ ID NO: 3)
MEQKLISEEDL SAG MANEGEEIEL TEFPETEKER KDEEKLSSCS
EETTNTSSSS GSDHVPVPIE VNVIIQNSSR TEDELQNSKE
IELTGFQGKL SSCSEETTAP SSSYSSKQAS VFIEENGDNE
TSTYRPQNVL TNLNSLYTTF EDARAQGKGM VRHKSEDLQS
FLEKYPPDFR KPKRDLSATW DPGMPTPPLP PRPANLGERQ
ASTVRLHVKE SNCKQPRERK ANERNIVKDL KRLENKINVI
ICLVVVILAV LLLVTVLSIL HIGMKS

2. Full Length TIP488 Y114A/Y127A (Aka YY->AA where Y[Tyr] to A[Ala] Mutation of STAT Binding Sites A [Y114] and B [Y127] in FIG. 3B)

(SEQ ID NO: 4)
MEQKLISEEDL SAG MANEGEEIEL TEFPETEKER KDEEKLSSCS
EETTNTSSSS GSDHVPVPIE VNVIIQNSSR TEDELQNSKE
IELTGFQGKL SSCSEETTAP SSSYSSKQAS VFIEENGDNE
TSTARPQNVL TNLNSLATTF EDARAQGKGM VRHKSEDLQS
FLEKYPPDFR KPKRDLSATW DPGMPTPPLP PRPANLGERQ
ASTVRLHVKE SNCKQPRERK ANERNIVKDL KRLENKINVI
ICLVVVILAV LLLVTVLSIL

### 3. YY->AA ΔN (N-Terminal Deletion)

(SEQ ID NO: 5)
MEQKLISEEDL SAG GDNE TSTARPQNVL TNLNSLATTF
EDARAQGKGM VRHKSEDLQS FLEKYPPDFR KPKRDLSATW
DPGMPTPPLP PRPANLGERQ ASTVRLHVKE SNCKQPRERK
ANERNIVKDL KRLENKINVI ICLVVVILAV LLLVTVLSIL
HIGMKS

### 4. YY->AA ΔH (Aliphatic Helix Deletion)

(SEQ ID NO: 6)
MEQKLISEEDL SAG MANEGEEIEL TEFPETEKER KDEEKLSSCS
EETTNTSSSS GSDHVPVPIE VNVIIQNSSR TEDELQNSKE
IELTGFQGKL SSCSEETTAP SSSYSSKQAS VFIEENGDNE
TSTARPQNVL TNLNSLATTF EDARAQGKGM VRHKSEDLQS
FLEKYPPDFR KPKRDLSATW DPGMPTPPLP PRPANLGERQ
ASTVRLHVKE SNCKQPRERK ANERN VI ICLVVVILAV
LLLVTVLSIL HIGMKS

### 5. YY->AA ΔN ΔH (N-Terminal Deletion+Aliphatic Helix Deletion)

(SEQ ID NO: 7)

MEQKLISEEDL SAG GDNE TSTARPQNVL TNLNSLATTF
EDARAQGKGM VRHKSEDLQS FLEKYPPDFR KPKRDLSATW
DPGMPTPPLP PRPANLGERQ ASTVRLHVKE SNCKQPRERK
ANERN VI ICLVVVILAV LLLVTVLSIL HIGMKS

### 6. Tip Minimal (N-Terminal Deletion+Aliphatic Helix Deletion+Transmembrane Deletion) [Also, Construct 2 FIG. 3B]

| (SEQ ID NO: 8) | |
|---|---|
| | MEQKLISEEDL SAG GDNE TSTARPQNVL TNLNSLATTF |
| | EDARAQGKGM VRHKSEDLQS FLEKYPPDFR KPKRDLSATW |
| | DPGMPTPPLP PRPANLGERQ ASTVRLHVKE SNCKQPRERK |
| | ANERN |

Expression of the various components of each peptide was confirmed by western blot using Jurkat T-cell lysates, as shown in FIG. 1C. LCK kinase activation by the TIP minimal viral protein was demonstrated in cells transduced with an empty vector (control) or a plasmid encoding the TIP minimal viral protein (protein 6, also referred to as “LCK activator”). LCK kinase activation was assessed by autophosphorylation (pY394). As shown in FIG. 1D, the amount of pY394 was enhanced in cells transfected with the TIP minimal viral protein compared to cells transfected with the empty vector. Expression was normalized against GAPDH. Taken together, these results demonstrate that the TIP minimal viral protein increases LCK kinase activity in CD8+ T-cells.

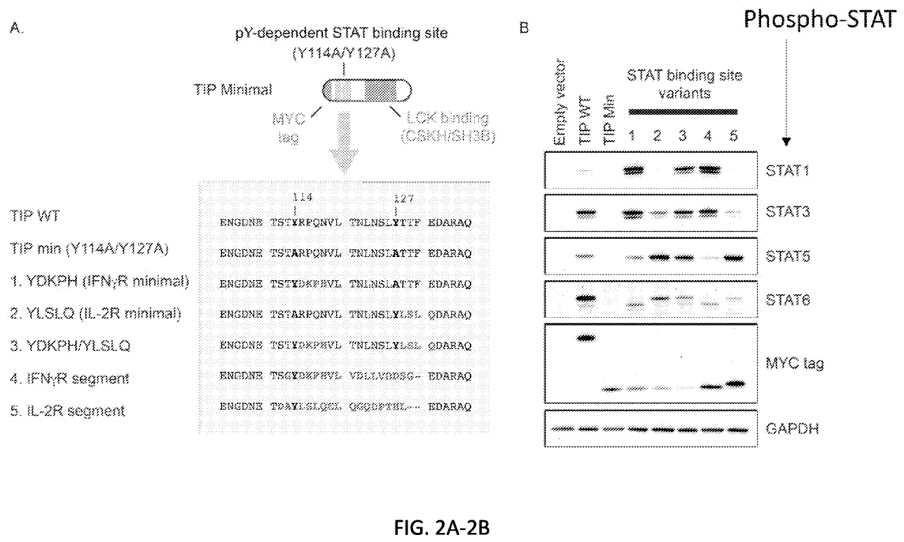

Next, various STAT binding sequences were inserted into the minimal TIP viral protein to generate bifunctional peptides. Exemplary STAT binding sequences were derived from cytokine receptors. Exemplary sequences are shown in FIG. 2A. The TIP WT protein in FIG. 2A corresponds to the TIP minimal viral protein identified and tested in FIG. 1. The TIP min (Y114A/Y127A) is a mutant minimal TIP viral protein modified to have a Y114A substitution and a Y127A substitution relative to the minimal TIP viral protein (TIP WT). Peptides 1-5 contain STAT binding regions derived from various cytokine receptors. Peptide 1 contains a minimal STAT binding region derived from IFNγR (YDKPH) (SEQ ID NO: 9). Peptide 2 contains a minimal STAT binding region derived from IL-2R (YLSLQ) (SEQ ID NO: 10). Peptide 3 contains a STAT binding region including both YDKPH (SEQ ID NO: 9) and YLSLQ (SEQ ID NO: 10). Peptide 4 contain a STAT binding region derived from IFNγR (GYDKPHVLVDLLVDDSG) (SEQ ID NO: 11). Peptide 5 contains a STAT binding region derived from IL-2R (DAYLSLQELQGQDPTHL) (SEQ ID NO: 12). The full sequences for exemplary bifunctional peptides containing the minimal TIP viral protein (e.g. containing an LCK binding region) and a STAT binding region are below. Sequences are annotated as follows:
MYC tag—MEQKLTSEEDL SAG (SEQ ID NO: 2)
LCK interaction sites: CSKH, SH3B
STAT binding site tyrosine residues A[Y114] and B[Y127] are italicized and underlined). Y to A mutations are similarly colored to denote position of mutated residue.

### Tip Minimal Y114 (Site “A”) [Construct 3 FIG. 3B]

| (SEQ ID NO: 13) | |
|---|---|
| | MEQKLISEEDL SAG GDNE TSTYRPQNVL TNLNSLATTF |
| | EDARAQGKGM VRHKSEDLQS FLEKYPPDFR KPKRDLSATW |
| | DPGMPTPPLP PRPANLGERQ ASTVRLHVKE SNCKQPRERK |
| | ANERN |

### Tip Minimal Y127 (Site “B”) [Construct 4 FIG. 3B]

| (SEQ ID NO: 14) | |
|---|---|
| | MEQKLISEEDL SAG GDNE TSTARPQNVL TNLNSLYTTF |
| | EDARAQGKGM VRHKSEDLQS FLEKYPPDFR KPKRDLSATW |
| | DPGMPTPPLP PRPANLGERQ ASTVRLHVKE SNCKQPRERK |
| | ANERN |

### Tip Minimal Y114/Y127 [Site “AB”, Construct 5 FIG. 3B]

| (SEQ ID NO: 15) | |
|---|---|
| | MEQKLISEEDL SAG GDNE TSTYRPQNVL TNLNSLYTTF |
| | EDARAQGKGM VRHKSEDLQS FLEKYPPDFR KPKRDLSATW |
| | DPGMPTPPLP PRPANLGERQ ASTVRLHVKE SNCKQPRERK |
| | ANERN |

### STAT1 Activator (IFNgR STAT Binding Site YDKPH (SEO ID NO: 9)) [Construct 6 FIG. 3B][Construct 1 FIG. 2AB]

| (SEQ ID NO: 16) | |
|---|---|
| | MEQKLISEEDL SAG GDNE TSTYDKPHVL TNLNSLATTF |
| | EDARAQGKGM VRHKSEDLQS FLEKYPPDFR KPKRDLSATW |
| | DPGMPTPPLP PRPANLGERQ ASTVRLHVKE SNCKQPRERK |
| | ANERN |

### STAT5 Activator (IL-2Rb STAT Binding Site YLSLQ (SEO ID NO: 10)) [Construct 7 FIG. 3B][Construct 2 FIG. 2AB]

| (SEQ ID NO: 17) | |
|---|---|
| | MEQKLISEEDL SAG GDNE TSTARPQNVL TNLNSLYLSLQ |
| | DARAQGKGM VRHKSEDLQS FLEKYPPDFR KPKRDLSATW |
| | DPGMPTPPLP PRPANLGERQ ASTVRLHVKE SNCKQPRERK |
| | ANERN |

### STAT1/5 Activator [Construct 8 FIG. 3B][Construct 3 FIG. 2AB]

| (SEQ ID NO: 18) | |
|---|---|
| | MEQKLISEEDL SAG GDNE TSTYDKPHVL TNLNSLYLSLQ |
| | DARAQGKGM VRHKSEDLQS FLEKYPPDFR KPKRDLSATW |
| | DPGMPTPPLP PRPANLGERQ ASTVRLHVKE SNCKQPRERK |
| | ANERN |

Expanded STAT binding sites corresponding to FIG. 2AB with Y(Tyr) denoted in underlined italics.

### IFNgR Segment

| (SEQ ID NO: 19) | |
|---|---|
| | MEQKLISEEDL SAG GDNE TSTGYDKPHVLVDLLVDDSGE |
| | DARAQGKGM VRHKSEDLQS FLEKYPPDFR KPKRDLSATW |
| | DPGMPTPPLP PRPANLGERQ ASTVRLHVKE SNCKQPRERK |
| | ANERN |

### IL-2R Segment

| (SEQ ID NO: 20) | |
|---|---|
| | MEQKLISEEDL SAG GDNE TSTDAYLSLQELQGQDPTHLE |
| | DARAQGKGM VRHKSEDLQS FLEKYPPDFR KPKRDLSATW |
| | DPGMPTPPLP PRPANLGERQ ASTVRLHVKE SNCKQPRERK |
| | ANERN |

STAT activation for each peptide was evaluated using western blot, as shown in FIG. 2B. The viral STAT binding sites were shown to broadly activate many STAT proteins. The STAT binding sites from endogenous cytokine receptors can be used to target specific STATs for activation. The IL-2 receptor STAT binding site (peptide 2 and peptide 5 in FIG. 2B) activates STAT5 and the IFNgR STAT binding sites (peptide 1 and peptide 4 in FIG. 2B) activates STAT1. Peptide 3, which contains both the IL-2 receptor and the IFNgR binding sites, activates both STAT5 and STAT1. The size of the STAT binding region was also compared (4 amino acids versus 10-12 amino acids).

Next, novel STAT activation profiles were developed. STAT activation of various peptides was assessed by western blot using Jurkat T-cell lysates. STAT activation as tested for the WT minimal TIP viral protein, LCK kinase only, viral stat (A) (construct 3 in FIG. 3B), viral STAT (B) (construct 4 in FIG. 3B), viral stat (AB) (construct 5 in FIG. 3B), an activator of STAT 1 (aSTAT1), an activator of STAT 5 (aSTAT5), an activator of STAT 1 and STAT 5 (aSTAT1/5), and an empty vector as a control. aSTAT1 is peptide 1 from FIG. 2, aSTAT5 is peptide 2 from FIG. 2, and a STAT1/5 is peptide 3 from FIG. 2. As shown in FIG. 3B, various STAT proteins are activated to different degrees by the different peptides tested, identifying novel STAT activation profiles for the different peptides. FIG. 3C summarizes the STAT activation profile for each tested peptide in a color-coded graph.

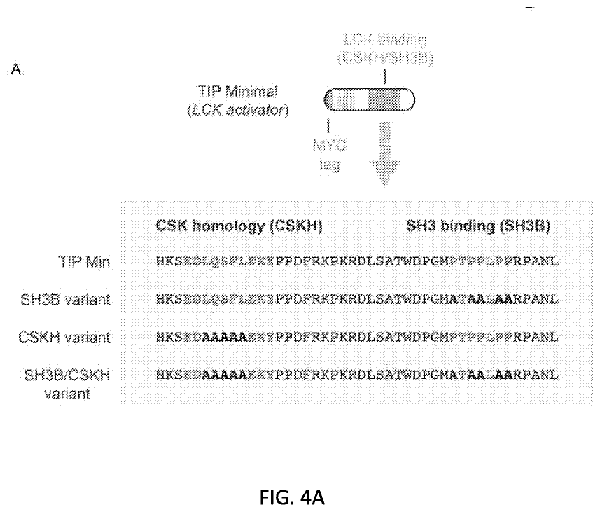

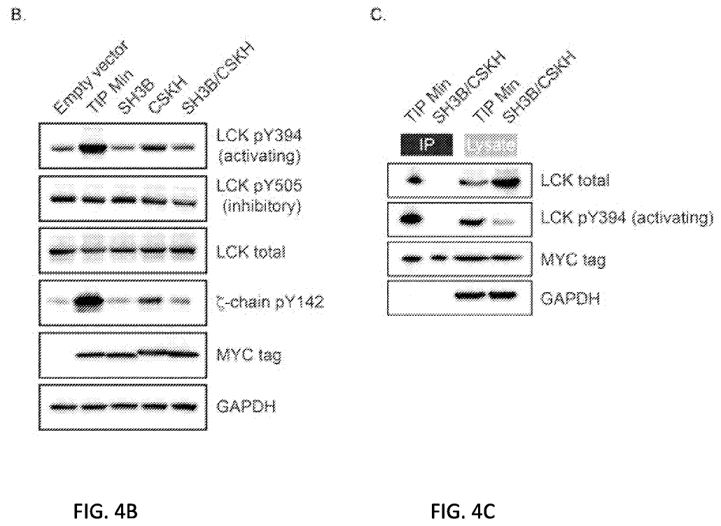

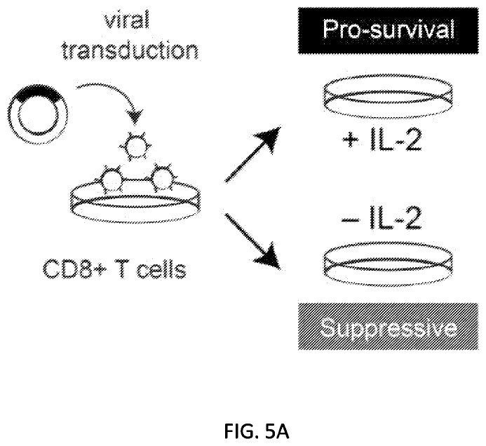

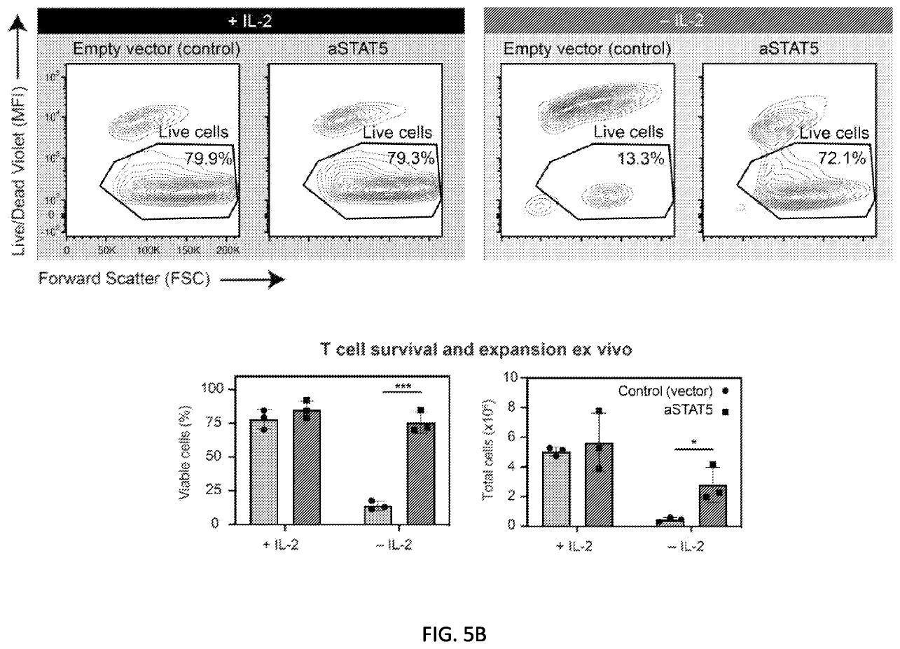

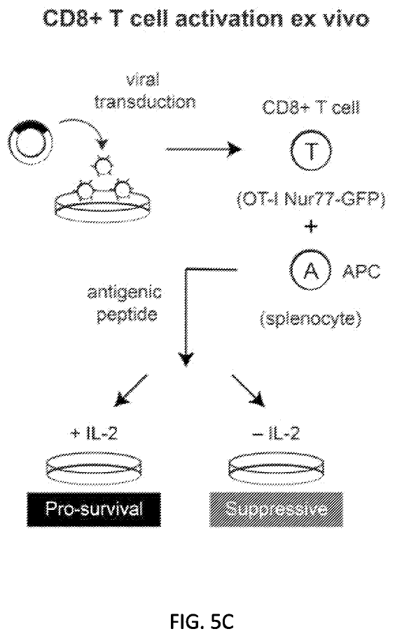

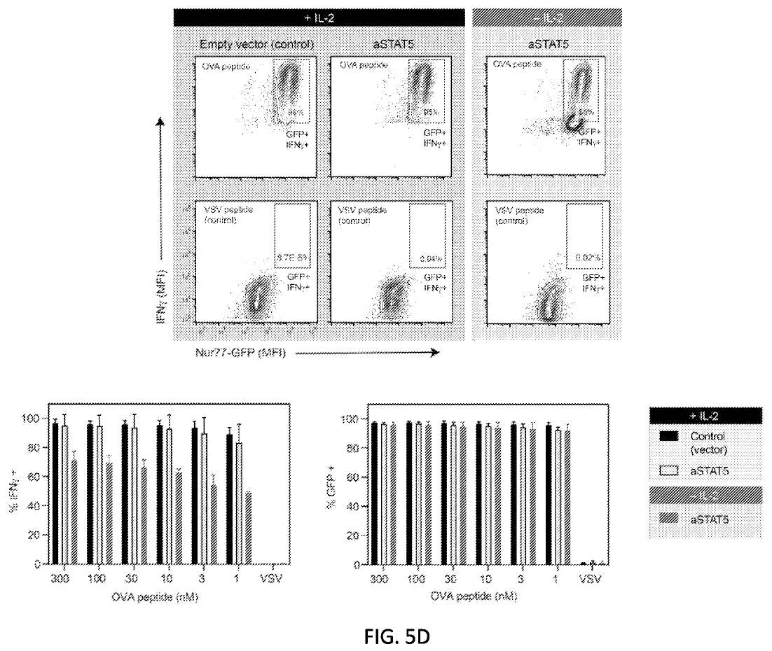

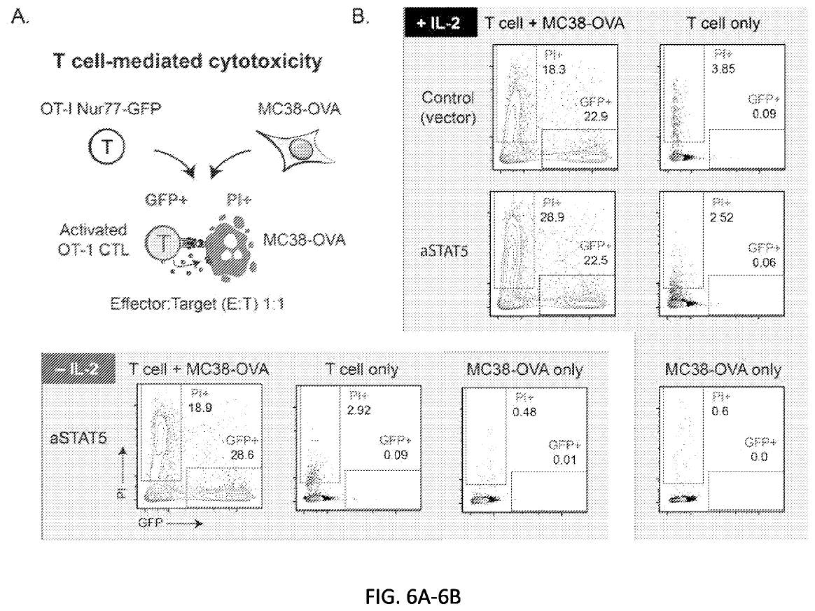

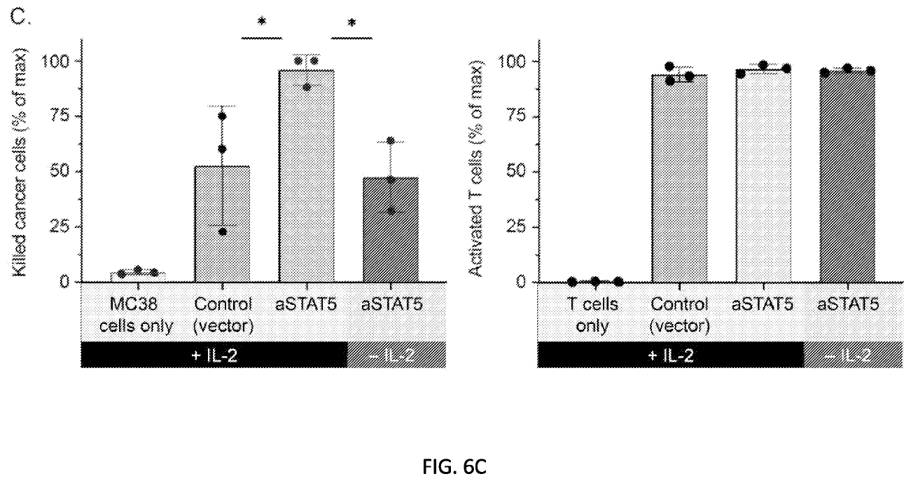

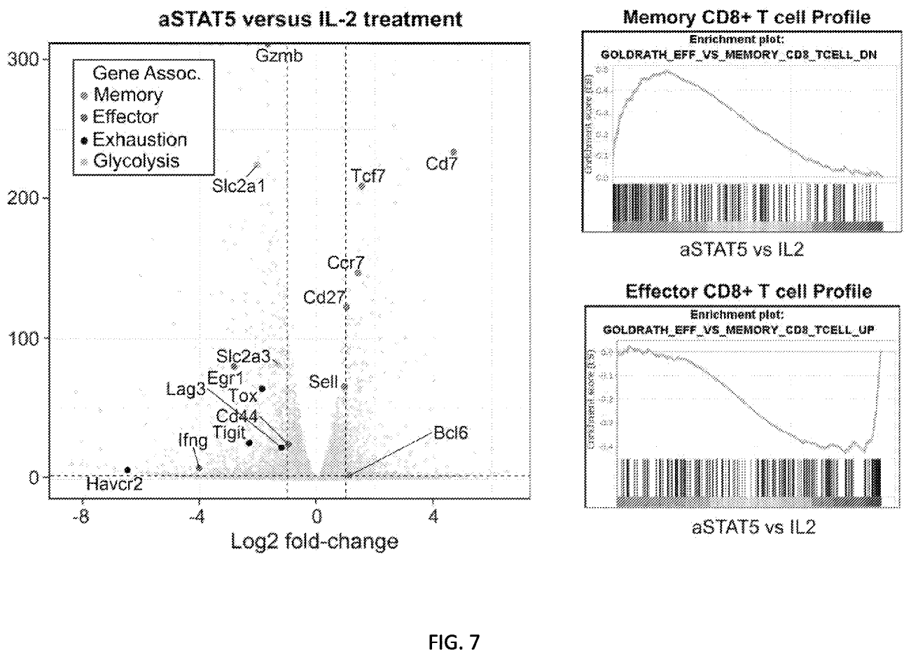

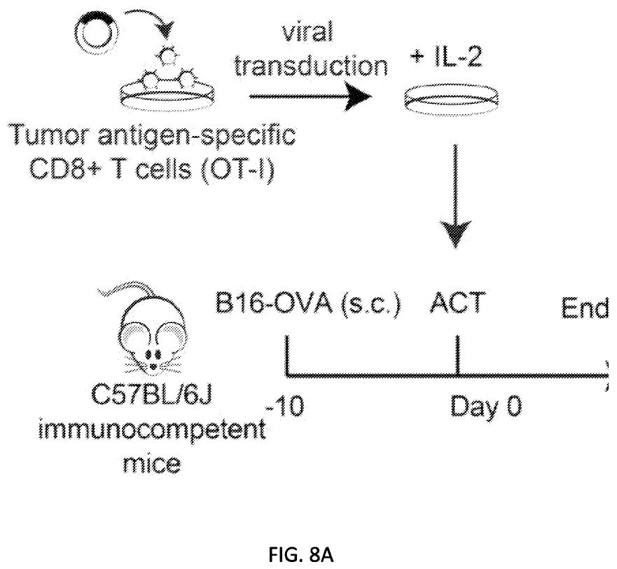

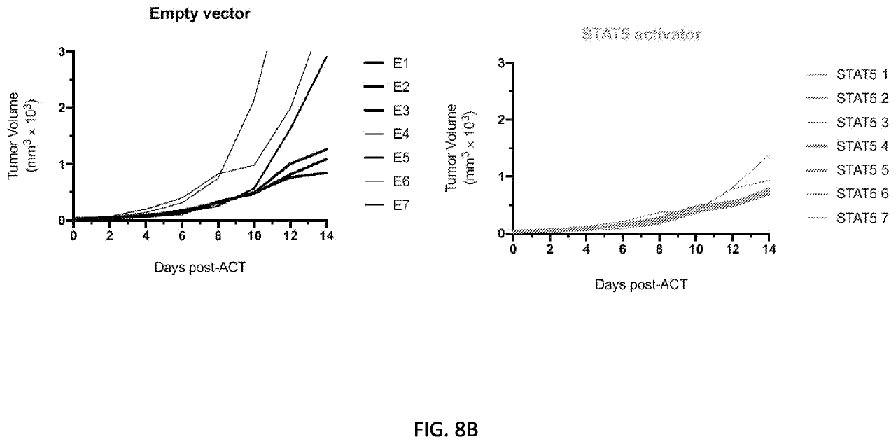

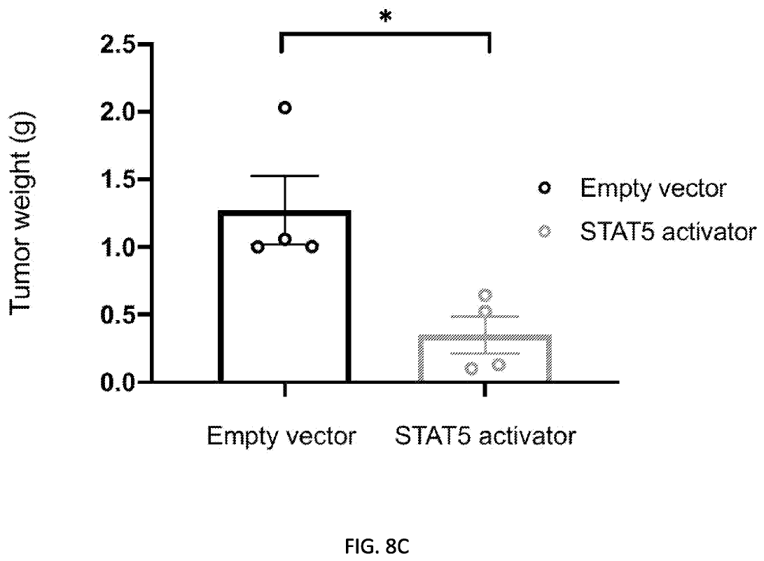

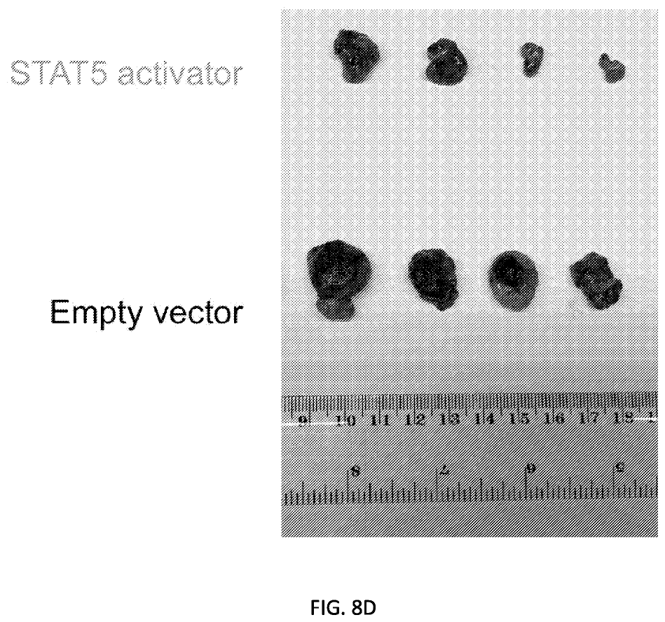

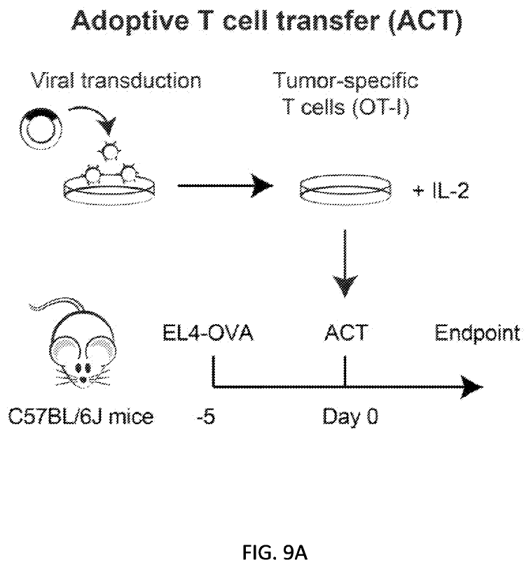

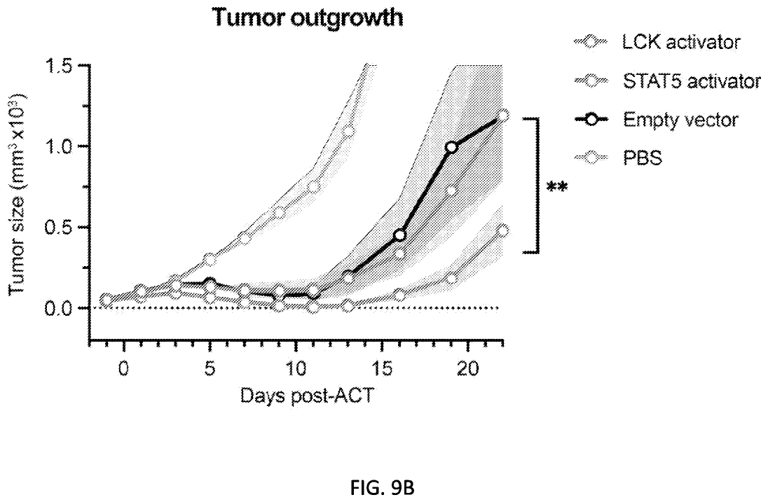

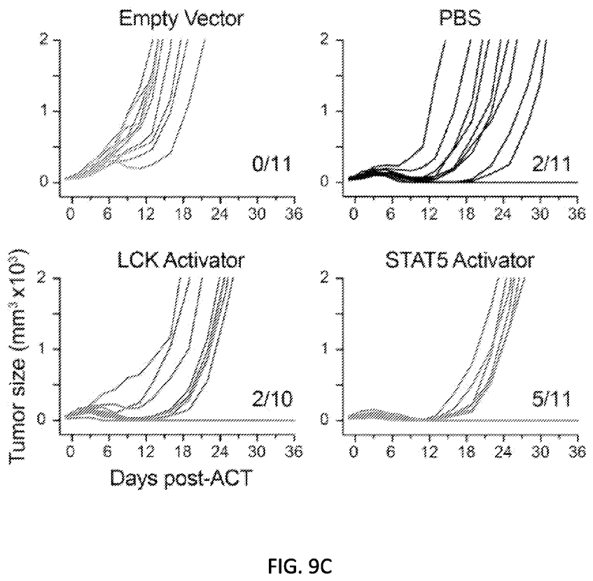

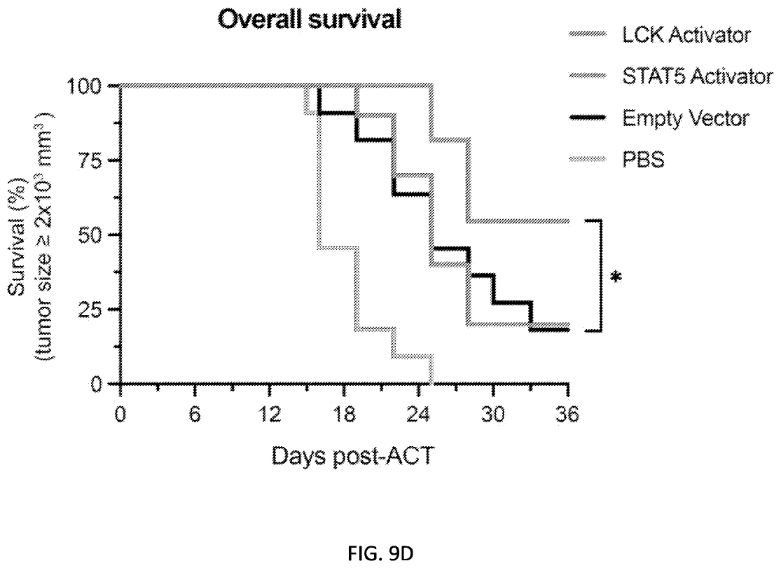

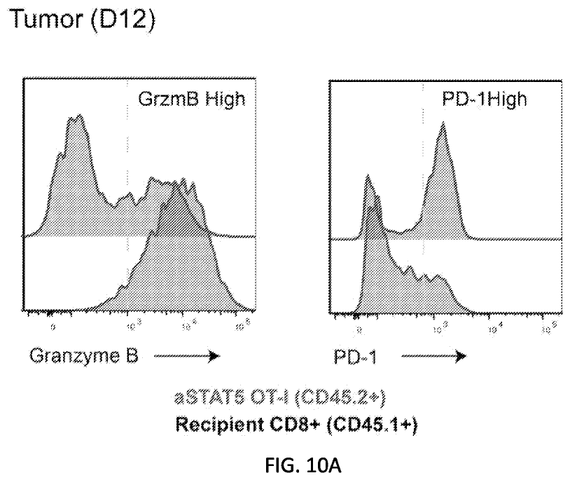

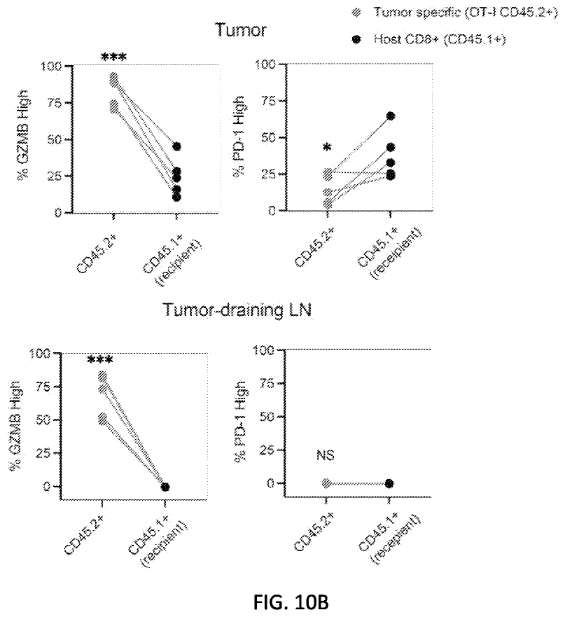

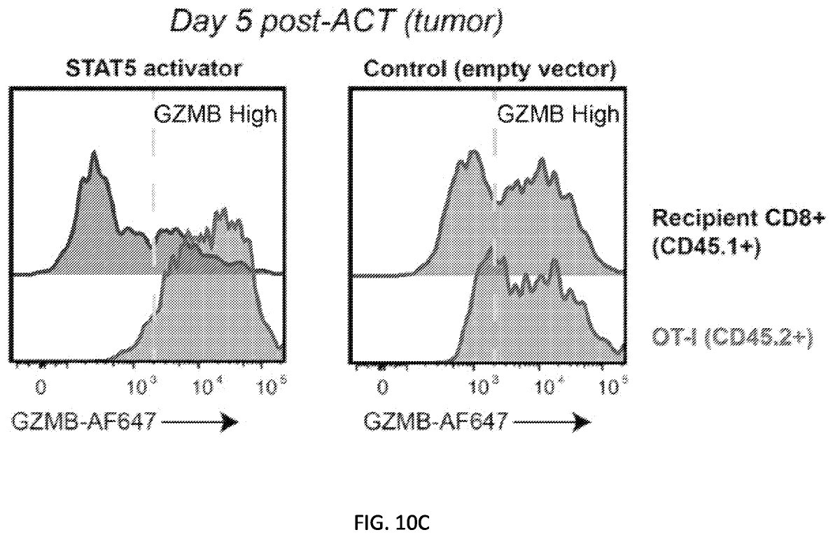

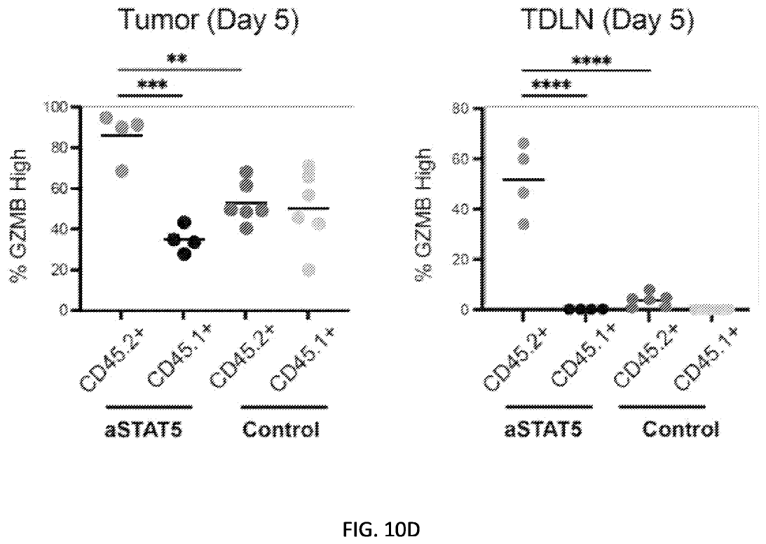

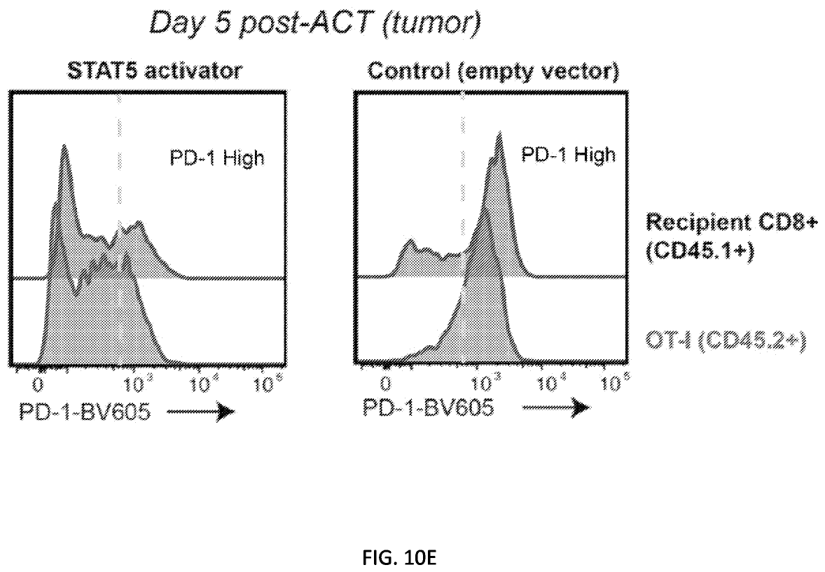

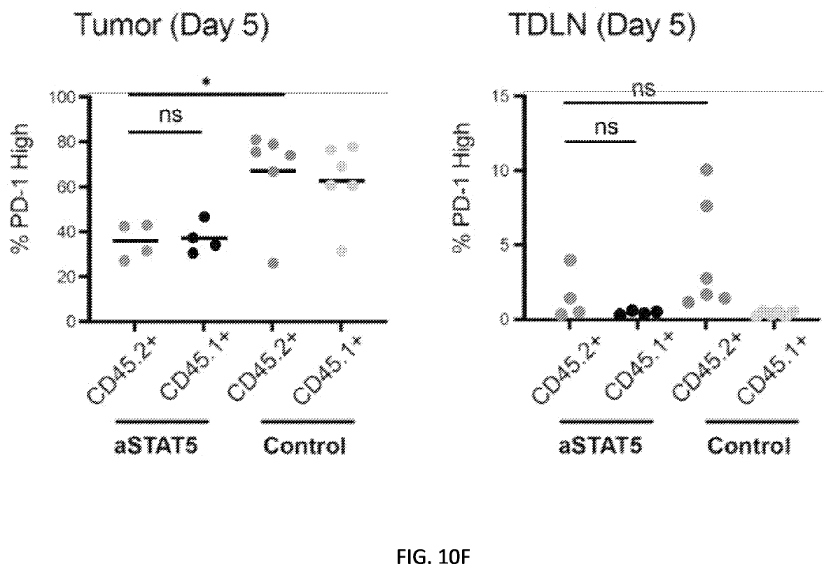

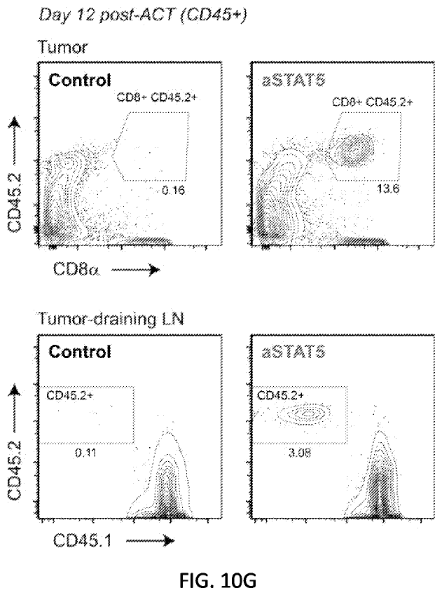

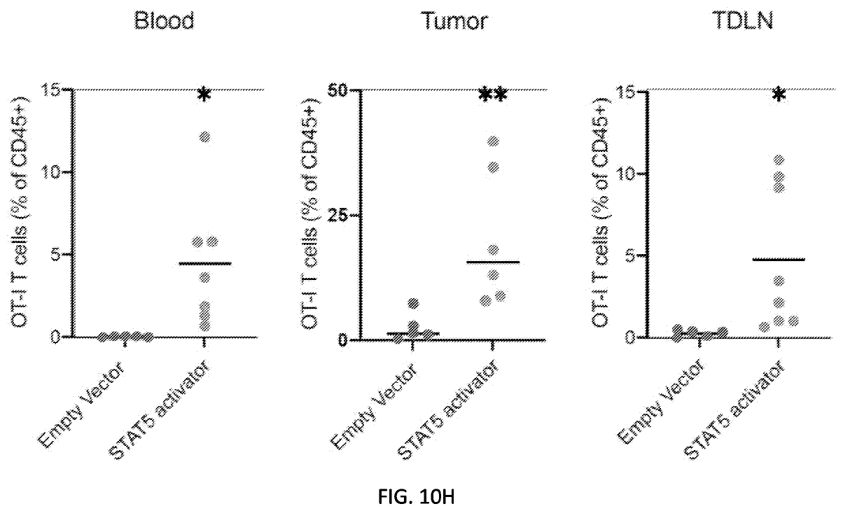

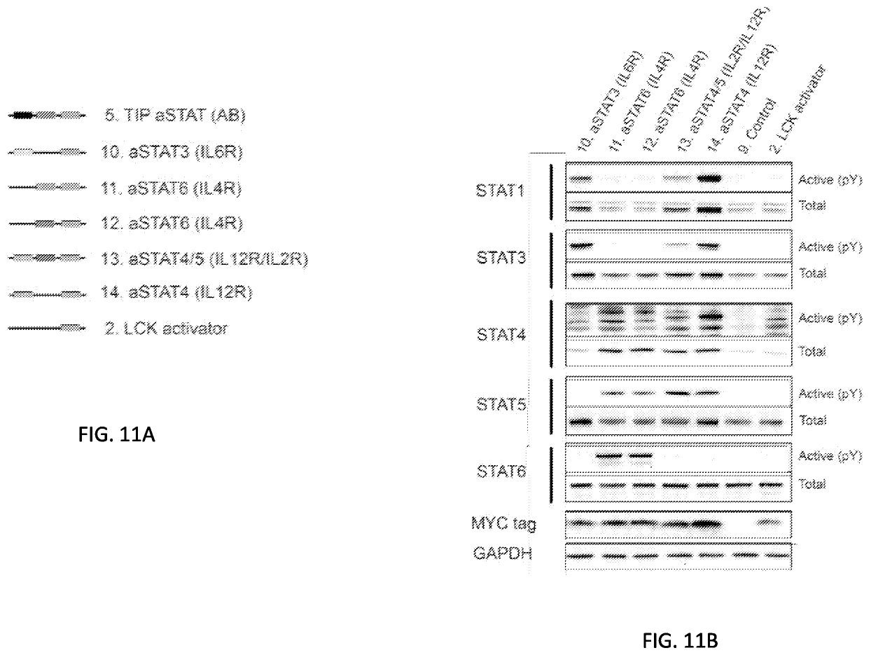

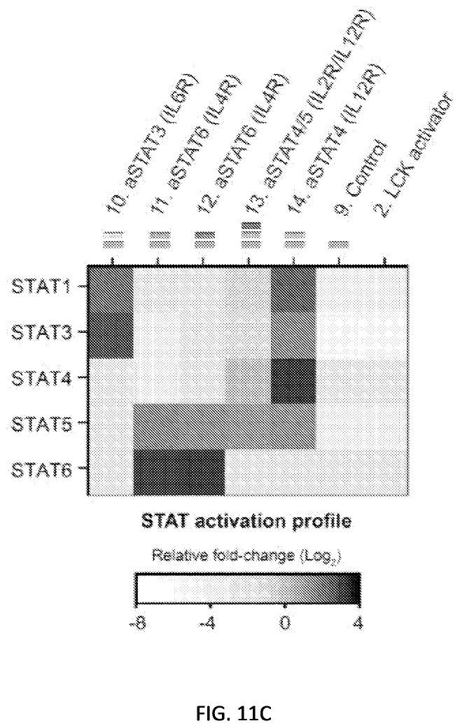

Mutations to CSKH and SH3B regions were made to assess the effect on LCK activity and binding to minimal peptide. Exemplary mutant sequences are shown in FIG. 4A. Jurkat T-cells were transfected with constructs encoding the mutant peptides and used to assess LCK activation and binding. Mutation of SH3B or CSKH in isolation dramatically reduces LCK auto phosphorylation (pY394) readout of kinase activity, as shown in FIG. 4B. Mutation of both sites disrupts TIP-mediated LCK activation. Immunoprecipitation/pulldown was performed to detect association (binding) of minimal TIP peptide to LCK kinase. Results are shown in FIG. 4C. A shown, SH3B/CSKH mutation disrupts binding in comparison to a minimal TIP peptide (left two lanes denote IP/pulldown).

Next, the STAT5 activator construct was used to determine whether a STAT activator can rescue or sustain T-cell survival in vivo. An schematic of an exemplary experiment conducted herein is shown in FIG. 5A. CD8+ T-cells were transduced with aSTAT5 or with empty constructs as a control, and cell survival and expansion was assessed in the presence and absence of exogenous IL-2 added to cell culture media. Results are shown in FIG. 5B. As shown, no difference in cell survival (assessed by % viable cells) or expansion (assessed by total number of cells) was observed for aSTAT5 expressing and control cells when IL-2 was added to culture media. Control cells demonstrated significantly reduced cell viability and significant reduction in total cell numbers when exogenous IL-2 was not added. In contrast, aSTAT5 expressing cells demonstrated no significant reduction in cell viability or total cell numbers in the absence of exogenous IL-2 compared to conditions where IL-2 was present in the cell culture media. Taken together, these data indicate that expression of aSTAT5 sustains T-cell survival in-vivo, even in the absence of exogenous IL-2.

It was next investigated whether aSTAT5 expressing T-cells retain cell function. A schematic of an exemplary experiment conducted herein is shown in FIG. 5C. T-cell function was assessed by the activation reporter Nur77-GFP and the intracellular cytokine IFNg. CD8+ T-cells were transduced with the aSTAT5 construct and the activation reporter Nur77-GFP. Antigen presenting cells (splenocytes) and an antigenic peptide (OVA peptide) or a control peptide (VSV peptide) were added to the culture. T-cell function was investigated in the presence and absence of exogenous IL-2. Results are shown in FIG. 5D. As shown, in the presence of exogenous IL-2, aSTAT5 expressing cells and displayed about 95% cell activation (measured by expression of both GFP and IFNg) in the presence of the antigenic OVA peptide, and control cells displayed about 96% activation, indicating no difference in activation between the two cell types under antigenic conditions with exogenous IL-2. In the absence of IL-2, aSTAT5 expressing cells displayed about 68% activation (measured by expression of both GFP and IFNg), indicating a substantial retention of cell function even in the absence of exogenous IL-2. Results were further evaluated by determining the % of IFNg positive cells and the % of GFP+ cells after activation with OVA peptide. As shown in the bar graphs, aSTAT5 expressing cells displayed no significant difference in the percentage of GFP+ cells following activation with the OVA peptide in the presence or absence of exogenous IL-2, demonstrating that expression of aSTAT5 enables T-cells to retain function even in the absence of exogenous IL-2.

To further evaluate T-cell function, T-cells transduced with the aSTAT5 construct and the activation reporter Nur77-GFP (OT-1 Nur77-GFP cells, or “OT-1” cells), were co-cultured with MC38-OVA cancer cells. A schematic of an exemplary experiment is shown in FIG. 6A. T-cell-medicated killing of cancer cells was quantified by imaging cytometry (quantification of propidium iodide positive (PI+) cells) and T-cell activation was evaluated by quantifying GFP positive cells. Results are shown in FIG. 6B and FIG. 6C. The results demonstrate that aSTAT5 expressing cells retain the capacity to recognize and kill cancer cells, even in the absence of exogenous IL-2.

CD8+ T-cell differentiation was next evaluated. CD8+ T-cells were transduced with aSTAT5, as described above, or treated with exogenous IL-2, and gene expression profiles were investigated. Results are shown in FIG. 7. It was observed that STAT5 activation produces a more memory-like T-cell phenotype than IL-2. The success of adoptive T-cell therapies (ACT) can hinge on capacity of transfused T-cells to self-renew and persist in the patient. Memory T-cells have a much higher potential to renew than more terminally differentiated effector T-cells. Accordingly, tumor-specific T-cells modified ex vivo to incorporate a STAT5 activator are predicted to be more effective in tumor models.

Experiments were conducted in-vivo to evaluate aSTAT5 activator efficacy in a tumorigenic mouse model. A schematic showing an exemplary experiment is shown in FIG. 8A. Tumor antigen-specific CD8+ T-cells were transduced with aSTAT5, LCK activator (TIP minimal protein, without the STAT binding sequence), PBS, or an empty vector and treated with exogenous IL-2. C57BL/6J immunocompetent mice were treated subcutaneously with B16-OVA cells at day −10. On day 0, adoptive cell transfer was conducted using the generated aSTAT5 expressing or control tumor antigen-specific CD8+ T-cells. Tumor volume was evaluated from days 1 to 14 after adoptive cell transfer. Results are shown in FIG. 8B. At day 16, tumors were harvested and weighed. Images of harvested tumors are shown in FIG. 8D, and weights are shown in FIG. 8C. As shown, mice that had received ACT using aSTAT5 cells showed significantly reduced tumor volume and significantly reduced tumor weight compared to mice that had received ACT using control cells. Taken together, this data shows that aSTAT5 increases anti-tumor activity of CD8+ T-cells in vivo. At day 16, no mice in the PBS group were alive and only 2 mice in the LCK activator group were alive.

Additional in-vivo experiments were conducted using a different mouse model. A schematic showing an exemplary experiment is shown in FIG. 9A. Tumor antigen-specific CD8+ T-cells were transduced with aSTAT5, LCK activator (TIP minimal protein, without the STAT binding sequence), PBS, or an empty vector and treated with exogenous IL-2. C57BL/6J immunocompetent mice were treated subcutaneously with EL4-OVA cells at day −5. At day 0, ACT was conducted. Tumor size was evaluated from days 0 to 22 post ACT. Results are shown in FIG. 9B. Mice treated with aSTAT5 showed significantly reduced tumor size compared to all other groups. Mice treated with LCK activator and empty vector showed similar tumor outgrowth. Plots showing individual mouse responses to each treatment are shown in FIG. 9C. Overall survival plots are shown in FIG. 9D. As shown, aSTAT5 mice displayed significantly increased overall survival compared to all other groups. Moreover, 5 out of 11 aSTAT5 mice showed a complete response to adoptive cell transfer. Data also demonstrate increased functionality in tumors due to STAT5 activation. As shown in FIG. 10A-10B, comparison of administered tumor specific T-cells (CD45.2+) to endogenous (CD45.1+) T-cells reveals high levels of Granzyme B, a marker associated with T-cell functionality, and reduced PD-1 Levels, which are associated with T-cell loss of function. Functionality was also assessed at day 5 post-ACT, where control tumor specific T-cells are still present. Results are presented in FIG. 10C-10F. STAT5 activation enhanced CD8+ T-cell persistence in tumors. As shown in FIG. 10G, 12 days post-ACT the percentage of CD45+ white blood cells were increased in aSTAT5 mice compared to empty vector control. Results were quantified in FIG. 10H.

Additional sequences were incorporated into the STAT activator platform to activate STAT 3, 4, or 6. Exemplary sequences for the STAT3 activator, STAT 6 activators, and STAT 4/5 activators are provided below. Sequences are annotated as follows:
MYC tag—MEQKLISEEDL SAG (SEQ ID NO: 2)
LCK interaction sites: CSKH, SH3B
STAT binding site tyrosine residues A[Y114] and B[Y127] are italicized and underlined). Y to A mutations are similarly identified to denote position of mutated residue.

### STAT3 Activator (IL-6R STAT Binding Site YRHQ (SEO ID NO: 26)) [Construct 10]

| (SEQ ID NO: 21) | |
|---|---|
| | MEQKLISEEDL SAG GDNE TSTYRHQNVL TNLNSLATTF |
| | EDARAQGKGM VRHKSEDLQS FLEKYPPDFR KPKRDLSATW |
| | DPGMPTPPLP PRPANLGERQ ASTVRLHVKE SNCKQPRERK |
| | ANERN |

### 11. STAT6 Activator (IL-4R STAT Binding Site YKAF (SEO ID NO: 27)) [Construct 11]

| (SEQ ID NO: 22) | |
|---|---|
| | MEQKLISEEDL SAG GDNE TSTARPQNVL TNLNSLYKAF |
| | EDARAQGKGM VRHKSEDLQS FLEKYPPDFR KPKRDLSATW |
| | DPGMPTPPLP PRPANLGERQ ASTVRLHVKE SNCKQPRERK |
| | ANERN |

### 12. STAT6 Activator (IL-4R STAT Binding Site YKPF (SEO ID NO: 28)) [Construct 12]

(SEQ ID NO: 23)
MEQKLISEEDL SAG GDNE TSTARPQNVL TNLNSLYKPF
EDARAQGKGM VRHKSEDLQS FLEKYPPDFR KPKRDLSATW
DPGMPTPPLP PRPANLGERQ ASTVRLHVKE SNCKQPRERK
ANERN

### 13. STAT4/5 Activator (IL-12R STAT Binding Site YLP (SEQ ID NO: 29) and IL-2Rb Site YISLQ (SEQ ID NO: 10)) [Construct 13]

| (SEQ ID NO: 24) | |
|---|---|
| | MEQKLISEEDL SAG GDNT HDGYLPSNIL TNLNSLYLSLQ |
| | DARAQGKGM VRHKSEDLQS FLEKYPPDFR KPKRDLSATW |
| | DPGMPTPPLP PRPANLGERQ ASTVRLHVKE SNCKQPRERK |
| | ANERN |

### 14. STAT4/5 Activator (IL-12R STAT Binding Site YLPSNIL (SEO ID NO: 29)) [Construct 14]

| (SEQ ID NO: 25) | |
|---|---|
| | MEQKLISEEDL SAG GDNT HDGYLPSNIL TNLNSLATTF |
| | EDARAQGKGM VRHKSEDLQS FLEKYPPDFR KPKRDLSATW |
| | DPGMPTPPLP PRPANLGERQ ASTVRLHVKE SNCKQPRERK |
| | ANERN |

Constructs are shown in FIG. 11A. Activation of STAT1, 3, 4, 5, and 6 was assessed by western blot, as shown in FIG. 11B. FIG. 11C summarizes the STAT activation profile for each tested peptide in a color-coded graph.

In sum, presented herein are STAT activators that promote T-cell survival and function. Such STAT activators can be used to promote T-cell efficacy in cancer. STAT activators find use in adoptive T-cell therapy (ACT), can be combined with chimeric antigen receptor T-cells (CAR T-cells) or patient-derived tumor infiltrating lymphocyte (TIL) therapies for treatment of cancer. STAT activators can be administered at the peptide level, or at the mRNA level, including to specific T-cells (e.g. isolated from a subject) or other immune cell populations. STAT activators can also be incorporated into CD4+ T-cells to further improve ACT.

We claim:

1. A bispecific peptide comprising at least one signal transducer and activator of transcription (STAT) binding region and at least one SRC family kinase binding region.

2. The bispecific peptide of claim 1, wherein the at least one STAT binding region comprises an amino acid sequence derived from one or more cytokine receptors.

3. The bispecific peptide of claim 1, wherein the at least one STAT binding region comprises an amino acid sequence derived from an interleukin-2 receptor (IL-2R), an interleukin-4 receptor (IL-4R), an interleukin-6 receptor (IL-6R), an interleukin-12 receptor (IL-12R), and/or an interferon gamma receptor (IFNgR).

4. The bispecific peptide of claim 1, wherein the at least one STAT binding region comprises one or more of YDKPH (SEQ ID NO: 9), YLSLQ (SEQ ID NO: 10), YRHQ (SEQ ID NO: 26), YKAF (SEQ ID NO: 27), YKPF (SEQ ID NO: 28), and YLPSNIL (SEQ ID NO: 29).

5. The bispecific peptide of claim 1, wherein the at least one STAT binding region comprises GYDKPHVLVDLLVDDSG (SEQ ID NO: 11).

6. The bispecific peptide claim 1, wherein the at least one STAT binding region comprises DAYLSLQELQGQDPTHL (SEQ ID NO: 12).

7. The bispecific peptide of any one of claims 1-6, wherein the at least one SRC family kinase binding region comprises at least one lymphocyte-specific protein tyrosine kinase (LCK) binding region.

8. The bispecific peptide of claim 7, wherein the at least one LCK binding region comprises an SH3 binding domain having at least 55% sequence identity to PTPPLPPRP (SEQ ID NO: 30) and/or a CSK homology domain having at least 50% sequence identity to VRHKSEDLQSFLEKYP (SEQ ID NO: 31).

9. The bispecific peptide of claim 8, wherein the at least one LCK binding region comprises an SH3 binding domain having at least 65% sequence identity to SEQ ID NO: 30 and/or a CSK homology domain having at least 75% sequence identity to SEQ ID NO: 31.

10. The bispecific peptide of claim 8, wherein the at least one LCK binding region comprises an SH3 binding domain having at least 75% sequence identity to SEQ ID NO: 30 and/or a CSK homology domain having at least 87.5% sequence identity to SEQ ID NO: 31.

11. The bispecific peptide of claim 8, wherein the at least one LCK binding region comprises an SH3 binding domain having at least 85% sequence identity to SEQ ID NO: 30 and/or a CSK homology domain having at least 93.5% sequence identity to SEQ ID NO: 31.

12. The bispecific peptide of claim 8, wherein the at least one LCK binding region comprises an SH3 binding domain comprising the sequence of SEQ ID NO: 30 and/or a CSK homology domain comprising the sequence of SEQ ID NO: 31.

13. A vector encoding the bispecific peptide of any one of claims 1-12.

14. A cell expressing the bispecific peptide of any one of claims 1-12 or the vector of claim 13.

15. The cell of claim 14, wherein the cell is an immune cell.

16. The cell of claim 15, wherein the immune cell is a natural killer (NK) cell, a T-cell, a B-cell, a macrophage, or a dendritic cell.

17. Use of the bispecific peptide of any one of claims 1-12, the vector of claim 13, or the cell of any one of claims 14-16 in a method of treating cancer in a subject.

18. A method of treating cancer in a subject, the method comprising providing to the subject a T-cell expressing a bispecific peptide comprising at least one signal transducer and activator of transcription (STAT) binding region and at least one SRC family kinase binding region.

19. The method of claim 18, wherein the at least one STAT binding region comprises an amino acid sequence derived from one or more cytokine receptors.

20. The method of claim 18, wherein the at least one STAT binding region comprises an amino acid sequence derived from an interleukin-2 receptor (IL-2R) and/or an amino acid sequence derived from an interferon gamma receptor (IFNgR).

21. The method of claim 18, wherein the at least one STAT binding region comprises one or more of YDKPH (SEQ ID NO: 9), YLSLQ (SEQ ID NO: 10), YRHQ (SEQ ID NO: 26), YKAF (SEQ ID NO: 27), YKPF (SEQ ID NO: 28), and YLPSNIL (SEQ ID NO: 29).

22. The method of claim 18, wherein the at least one STAT binding region comprises GYDKPHVLVDLLVDDSG (SEQ ID NO: 11).

23. The method of claim 18, wherein the at least one STAT binding region comprises DAYLSLQELQGQDPTHL (SEQ ID NO: 12).

24. The method of any one of claims 18-23, wherein the at least one SRC family kinase binding region comprises at least one lymphocyte-specific protein tyrosine kinase (LCK) binding region.

25. The method of claim 24, wherein the at least one LCK binding region comprises an SH3 binding domain having at least 55% sequence identity to PTPPLPPRP (SEQ ID NO: 30) and/or a CSK homology domain having at least 50% sequence identity to VRHKSEDLQSFLEKYP (SEQ ID NO: 31).

26. The method of claim 25, wherein the at least one LCK binding region comprises an SH3 binding domain having at least 65% sequence identity to SEQ ID NO: 30 and/or a CSK homology domain having at least 75% sequence identity to SEQ ID NO: 31.

27. The method of claim 25, wherein the LCK binding region comprises an SH3 binding domain having at least 75% sequence identity to SEQ ID NO: 30 and/or a CSK homology domain having at least 87.5% sequence identity to SEQ ID NO: 31.

28. The method of claim 25, wherein the LCK binding region comprises an SH3 binding domain having at least 85% sequence identity to SEQ ID NO: 30 and/or a CSK homology domain having at least 93.5% sequence identity to SEQ ID NO: 31.

29. The method of claim 25, wherein the LCK binding region comprises an SH3 binding domain comprising the sequence of SEQ ID NO: 30 and/or a CSK homology domain comprising the sequence of SEQ ID NO: 31.

30. The method of any one of claims 18-29, wherein the T-cell expressing the bispecific peptide is co-administered to the subject in combination with exogenous IL-2.

31. The method of any one of claims 18-29, wherein the T-cell expressing the bispecific peptide is administered to the subject without co-administration of exogenous IL-2.

32. The method of any one of claims 18-31, wherein the subject is a human.
